(19) United States
(12) Patent Application Publication
(10) Pub. No.: US 2019/0100794 A1
(43) Pub. Date: Apr. 4, 2019

(54) Blood matrix metalloproteinase 9 (MMP9) is a predictive biomarker for cardiac diseases and disorders

(71) Applicant: University of Rochester, Rochester, NY (US)

(72) Inventors: Scott J. Cameron, Rochester, NY (US); Frederick Ling, Pittsford, NY (US)

(21) Appl. No.: 16/136,549

(22) Filed: Sep. 20, 2018

Related U.S. Application Data
(60) Provisional application No. 62/560,805, filed on Sep. 20, 2017.

(51) Int. Cl. C12Q 1/6851; A61P 9/00; A61K 31/50

(52) U.S. Cl. CPC C12Q 1/6851; A61P 9/00; A61K 31/50; C12Q 2600/158; C12Q 2600/112; C12Q 2600/118; C12Q 2600/106

(57) ABSTRACT

The present invention relates to compositions and methods useful for the assessment, diagnosis, and treatment of myocardial infarctions.

### CROSS-REFERENCE TO RELATED APPLICATIONS

This application is a U.S. non-provisional patent application that is entitled to priority to U.S. Provisional Application No. 62/560,805, filed Sep. 20, 2017, which is incorporated by reference herein in its entirety.

### STATEMENT REGARDING FEDERALLY SPONSORED RESEARCH OR DEVELOPMENT

This invention was made with government support under HL128856 and HL120200 awarded by National Institutes of Health. The government has certain rights in the invention.

### BACKGROUND OF THE INVENTION

More than fifty years ago, it was discovered that aspirin inhibits platelet activation by preventing endogenous thromboxane synthesis. It was also discovered that aspirin attenuates adenosine diphosphate (ADP) secretion from platelet dense granules—a mechanism to amplify platelet activation via the P2Y₁₂ receptor (Born G V, et al., 1963, J Physiol, 168:178-95; O'Brien J R., 1968, Lancet, 1:779-83; Weiss H J, et al., 1968, J Clin Invest, 47:2169-80.). Based upon those initial discoveries, dual antiplatelet therapy (DAPT), consisting of aspirin and a P2Y₁₂ receptor antagonist, showed efficacy in the treatment of ST-Elevation Myocardial Infarction (STEMI) (Yusuf S, et al., 2001, N Engl J Med, 345:494-502). Societal guidelines for treating myocardial infarction (M.I.) recommend 12 months of Dual Antiplatelet Therapy (DAPT) following any STEMI or Non-ST-Elevation Myocardial Infarction (NSTEMI) (Bittl J A, et al., 2016, J Am Coll Cardiol, 68:1116-39; Damman P, et al., 2017, Neth Heart J, 25:181-185). This recommendation is based on the premise that the biological processes in platelets prior to and following STEMI and NSTEMI share pathophysiological similarities (Montalescot G, et al., 2007, Eur Heart J, 28:1409-17).

Failure of platelet P2Y₁₂ antagonists to have their anticipated effect in some patients has been attributed to single nucleotide polymorphisms (SNPs) in genes encoding enzymes responsible for P2Y₁₂ antagonist metabolism (Carlquist J F, et al., 2013, Thromb Haemost, 109:744-54; Viviani Anselmi C, et al., 2013, JACC Cardiovasc Interv, 6:1166-75). Bhatt et al. recently demonstrated that enteric-coated aspirin has delayed absorption and limited antiplatelet efficacy compared with non-enteric coated aspirin (Bhatt D L, et al., 2017, J Am Coll Cardiol, 69:603-612). These studies suggest a personalized approach to antiplatelet therapy ought to be considered. Longitudinal studies examining patients treated for M.I. acknowledge that short term mortality in patients with STEMI is greater compared to NSTEMI. Paradoxically, long-term mortality for NSTEMI is reported to be greater in spite of appropriate utilization of cardiac catheterization and revascularization in these patients (Fox K A, et al., 2007, JAMA, 297:1892-900). Thus, beyond the initial thrombotic event, there may be a fundamental difference in biological processes in STEMI and NSTEMI (Chan M Y, et al., 2009, Circulation, 119:3110-7).

Whether circulating platelets in diseased conditions are phenotypically and therefore functionally similar to platelets in healthy subjects in whom preclinical studies for antiplatelet agents are conducted is an under-explored area of investigation (Nicholson N S, et al., 1998, Am Heart J, 135:S170-8). It has been recently reported that platelet ERK5 alters the platelet phenotype in a murine model of thrombotic and ischemic disease and in platelets exposed to redox stress (Cameron S J, et al., 2015, Circulation, 132:47-58; Yang M, et al., 2017, Blood, 129:2917-2927). Hu et al. recently demonstrated in both rodent models and in humans that diabetes alters signaling properties of the P2Y₁₂ receptor and post-receptor signaling pathway with subsequent resistance to antiplatelet medications (Hu L, et al., 2017, Circulation, 136(9): 817-833). These studies suggest greater attention should be paid to changes in post-receptor signal transduction pathways in the platelet in diseased conditions. Such studies could aid the discovery of novel targets inside platelets to abrogate dysregulated platelet signaling in thrombotic disease.

Thus, there remains a need in the art for an improved understanding of platelets and their role in cardiac diseases and disorders, and for the identification of biomarkers to enable distinction between STEMI and NSTEMI. The present invention addresses this unmet need.

### SUMMARY OF THE INVENTION

In one aspect, the present invention provides a method of diagnosing ST-elevation myocardial infarction (STEMI) in a subject, the method comprising: detecting the level of MMP9 in a biological sample obtained from the subject; comparing the level of MMP9 in the biological sample to a control level of MMP9; and diagnosing the subject with STEMI when MMP9 is elevated in the biological sample as compared to the control level. In one embodiment, the biological sample comprises plasma.

In one embodiment, the subject is diagnosed with STEMI when the level of MMP9 in the sample is increased by 1.5-fold or greater as compared to the level of MMP9 in a subject or population having non-ST-elevated myocardial infarction (NSTEMI).

In one embodiment, the subject is diagnosed with STEMI when the level of MMP9 in the sample is increased by about 2.4 fold or greater as compared to the level of MMP9 in a healthy control subject or population not having myocardial infarction.

In one embodiment, the method further comprises administering a STEMI treatment to the subject. In one embodiment, the STEMI treatment comprises at least one treatment selected from the group consisting of angioplasty, stent placement, coronary artery bypass surgery, administration of one or more thrombolytic drugs, and administration of one or more thromboxane and P2Y₁₂ receptor antagonists.

In one aspect, the present invention provides a method of diagnosing non-ST-elevation myocardial infarction (NSTEMI) in a subject, the method comprising: detecting the level of MMP9 in a biological sample obtained from the subject; comparing the level of MMP9 in the biological sample to a control level of MMP9; and diagnosing the subject with NSTEMI when MMP9 is differentially expressed in the biological sample as compared to the control level. In one embodiment, the biological sample comprises plasma.

In one embodiment, the subject is diagnosed with NSTEMI when the level of MMP9 in the sample is decreased by greater than about 1.5-fold relative to the level of MMP9 in a subject or population having STEMI.

In one embodiment, the subject is diagnosed with NSTEMI when the level of MMP9 in the sample is increased by about 1.4 fold or greater as compared to the level of MMP9 in a healthy subject or population not having myocardial infarction.

In one embodiment, the subject is diagnosed with NSTEMI when a) the level of MMP9 in the sample is decreased by about 1.5 fold or greater as compared to the level of MMP9 in a subject or population having ST-elevated myocardial infarction (STEMI), and b) the level of MMP9 in the sample is increased by about 1.4 fold or greater as compared to the level of MMP9 in a healthy subject or population not having myocardial infarction.

In one embodiment, the method further comprises administering a NSTEMI treatment to the subject. In one embodiment, the NSTEMI treatment comprises at least one treatment selected from the group consisting of administration of one or more blood thinners, administration of one or more thromboxane, PAR1, or P2Y₁₂ receptor antagonists, angioplasty, stent placement, and coronary artery bypass surgery.

In one aspect, the present invention provides a kit for distinguishing between STEMI and NSTEMI, the kit comprising a reagent for measuring the level of MMP9 in a biological sample of a subject. In one embodiment, the biological sample comprises plasma.

In one aspect, the present invention provides a method of treating STEMI in a subject who has been identified as having a differentially expressed level MMP9, comprising administering an effective STEMI treatment to the subject. In one embodiment, the differentially expressed level of MMP9 comprises: an about 1.5 fold increase as compared to the level of MMP9 in a subject or population having non-ST-elevated myocardial infarction (NSTEMI); or an about 2.4. fold increase as compared to the level of MMP9 in a healthy subject or population not having myocardial infarction.

In one aspect, the present invention provides a method of treating NSTEMI in a subject who has been identified as having a differentially expressed level of MMP9, comprising administering an effective NSTEMI treatment to the subject. In one embodiment, the differentially expressed level of MMP9 comprises: an about 1.5 fold decrease as compared to the level of MMP9 in a subject or population having ST-elevated myocardial infarction (STEMI); or an about 1.4. fold increase as compared to the level of MMP9 in a healthy subject or population not having myocardial infarction.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts results from example experiments, summarizing patient demographics. 40 subjects with M.I. were studied (20 STEMI, 20 NSTEMI). Patient demographics are noted. The first venous blood sample was used for most analyses. *p=0.03, otherwise no significant difference in continuous variables between groups. For race: C=Caucasian, B=Black, A=Asian. CKD=chronic kidney disease. LVEF=left ventricular ejection fraction. cTnT=cardiac troponin T. LDL=low density lipoprotein. HDL=high density lipoprotein. RCA=right coronary artery. LCx=left circumflex coronary artery. LAD: left anterior descending coronary artery. CABG=coronary artery bypass graft.

FIG. 2A through FIG. 2C depict results from example experiments, demonstrating platelet reactivity in patients with STEMI and NSTEMI compared to control subjects. Platelets were isolated and examined basally (0) or after stimulation with agonists for (FIG. 2A) the P2Y₁₂ receptor (ADP), (FIG. 2B) the thromboxane receptor (U46619), and (FIG. 2C) protease-activated receptor 1 (PAR1) for 15 mins and activation assessed by FACS by P-selectin expression, Mean Fluorescence Intensity (MFI)±SEM, all performed in quadruplicate in each group, n=12-17. Red=individual STEMI responses. Blue=individual NSTEMI responses. Black broken line=normal volunteer subjects not taking aspirin.

FIG. 3A through FIG. 3C depict results from example experiments, demonstrating platelet reactivity in patients with STEMI is different from NSTEMI. All patients were given 325 mg aspirin at least 30 minutes prior to blood draw. Platelets were isolated and examined basally (0) or after stimulation with agonists for (FIG. 3A) the P2Y₁₂ receptor (ADP), (FIG. 3B) the thromboxane receptor (U46619), and (FIG. 3C) Protease-Activated Receptor 1 (PAR1) for 15 mins and activation assessed by FACS by P-selectin expression, Mean Fluorescence Intensity (MFI)±SEM, all performed in quadruplicate in each group, n=12-17. *p<0.05 and **P<0.01 between STEMI and NSTEMI.

FIG. 4A and FIG. 4B depict results from example experiments, demonstrating platelet ERK5 activation in STEMI and NSTEMI and that ERK5 inhibition prevents platelet activation. (FIG. 4A) All patients were given 325 mg aspirin at least 30 minutes prior to blood draw. Platelets were isolated from healthy volunteer subjects (control) or from patients with STEMI or NSTEMI. ERK5 activation was assessed by Western blotting using a phosphor-specific antibody (P-ERK5). A pan-ERK5 antibody was used to detect total ERK5 and the ratio (P-ERK5/ERK5) was used to report ERK5 activity as mean±SEM, n=10-13. *p<0.05 control vs. STEMI, **p=0.01 control vs NSTEMI. The molecular weight is indicated in kiloDaltons (kDa). (FIG. 4B) All patients were given 325-mg aspirin at least 30 minutes before blood draw. Platelets were isolated from healthy volunteer subjects (control, white) or from patients with NSTEMI (black) and incubated with BIX 02189 (0-100 μM, 30 minutes) before platelet stimulation with TRAP6 (10 μM, 15 minutes). Platelet activation was assessed by fluorescence-activated cell sorting by P-selectin expression, mean fluorescence intensity (MFI)±SEM, all performed in quadruplicate in each group, n=4. *P<0.05 between NSTEMI/TRAP6 and NSTEMI/TRAP6+BIX 02189 and **P<0.05 control/TRAP6 and control/TRAP6+BIX 02189.

FIG. 5A through FIG. 5C depict results from example experiments, demonstrating platelet biomarker evaluation in STEMI and NSTEMI. All patients were given 325 mg aspirin at least 30 minutes prior to blood draw. Platelets were isolated from healthy volunteer subjects (control) or from patients with STEMI or NSTEMI. (FIG. 5A) MMP9 protein content was assessed by Western blotting. (FIG. 5B) MMP activity was assessed by gel zymography. (FIG. 5C) TIMP1 protein expression was assessed by Western blotting. GAPDH is a loading control. Protein expression or MMP activity are represented as mean±SEM (n=5-19). *p<0.05 vs control, **p<0.01 vs control. The molecular weight is indicated in kiloDaltons (kDa). MMP=matrix metalloproteinase. TIMP=tissue inhibitor of MMP.

FIG. 6A through FIG. 6C depict results from example experiments, demonstrating plasma MMP activity in STEMI and NSTEMI. All patients were given 325 mg aspirin at least 30 minutes prior to blood draw. Plasma was isolated and (FIG. 6A) MMP activity was assessed by gel zymography and (FIG. 6B) displayed graphically as % active MMP9 detected in plasma (n=15 samples for STEMI, n=9 samples for NSTEMI). (FIG. 6C) Plasma, platelets, or coronary thrombus were examined by zymography in the same patient with an acute inferior STEMI attributed to a right coronary artery (RCA) aspirated thrombus. MMP9 appears to be the most active gelatinase in these samples.

FIG. 7A through FIG. 7C depict results from example experiments, demonstrating plasma biomarker evaluation in STEMI and NSTEMI. Blood was taken from patients immediately upon presentation with STEMI or NSTEMI and compared to healthy volunteer subjects. Plasma was isolated and biomarker concentration was evaluated by ELISA. Patients with STEMI or NSTEMI were given 325 mg aspirin at least 30 minutes prior to blood draw. Data are represented as mean±SEM (n=20 each group) for (FIG. 7A) Plasma MMP9, *p<0.05 vs. control **p<0.01 vs. control and p=0.025 STEMI vs. NSTEMI. (FIG. 7B) Plasma TIMP1, p=NS between all groups. (FIG. 7C) Plasma MMP9/TIMP1 ratio *p<0.05 vs. control p<0.001 vs. control and p=0.015 STEMI vs. NSTEMI. The mean plasma concentration of each marker is shown below each graph. ELISA=Enzyme-Linked Immunosorbant Assay.

FIG. 8A and FIG. 8B depict results from example experiments, demonstrating plasma biomarker evaluation in STEMI and NSTEMI by ROC analysis. Blood was taken from patients immediately upon presentation with STEMI or NSTEMI and compared to healthy volunteer subjects. Patients with STEMI or NSTEMI were given 325 mg aspirin at least 30 minutes prior to blood draw. ROC analysis was used to determine the performance of (FIG. 8A) plasma MMP9 from the first blood sample obtained from the patient. *p<0.0001 predicting STEMI. § p=0.03 predicting NSTEMI and (FIG. 8B) Plasma MMP9 and cTnT in distinguishing between STEMI and NSTEMI, *p=0.00⁶ for MMP9 between groups and ¶ p=0.08 for cTnT between groups. AUC=area under curve. cTnT=cardiac troponin T.

FIG. 9 depicts results from example experiments, demonstrating plasma TxB2 concentration. Blood was taken from patients immediately upon presentation with STEMI or NSTEMI and compared to healthy volunteer subjects. Each patient had been given 325 mg aspirin at least 30 mins prior to the blood draw. Plasma was isolated and biomarker concentration was evaluated by ELISA. Plasma TxB2 concentration served as a surrogate marker to assess for differences in efficacy of aspirin between groups, comparing this to healthy volunteer subjects (control) not taking aspirin. Data are represented as mean±SEM (n=10-20 in each group). *p<0.05 vs. control, **p<0.001 vs. control and, p=NS STEMI vs. NSTEMI. ELISA=Enzyme-Linked Immunosorbant Assay.

FIG. 10A and FIG. 10B depict results from example experiments, demonstrating platelet MMP9 expression. Isolated washed platelet-rich plasma from five healthy volunteers was assessed for MMP9 protein content by Western blotting. The isolated, washed platelet rich plasma (PRP) was divided into two with a CD45 or control IG depletion step applied to one sample. (FIG. 10A) Western blotting was conducted using antibodies for cell marker proteins: CD45 (WBC), CD41 (platelets). A human macrophage cell line (THP1) was used in lane 1 was as a positive control for WBC and is CD45(+)/CD41(−). (FIG. 10B) MMP9 content in PRP before CD45 depletion (+) and after CD45 depletion (−). Proteins were quantified by densitometry and reported as mean MMP9/CD41±SEM above each graph (n=5, p=0.36 between groups). GAPDH is a loading control. * immunoreactive, proteolytic fragment.

### DETAILED DESCRIPTION

The present invention relates to the discovery that the expression patterns of certain genes or otherwise expression levels of some biomarkers are associated with the diagnosis and assessment of NSTEMI and STEMI. Thus, in various embodiments described herein, the methods of the invention relate to methods of diagnosing a subject as having NSTEMI or STEMI, methods for differentiating NSTEMI from STEMI, and methods of treating a desired condition and methods of altering treatment in a subject.

In some embodiments, biomarkers associated with NSTEMI or STEMI are up-regulated, while in other embodiments, the biomarkers associated with NSTEMI or STEMI are down-regulated. Thus, the invention relates to compositions and methods useful for the detection and quantification of biomarkers, including RNA, protein and other biomarkers, for the diagnosis, assessment, and characterization of NSTEMI or STEMI in a subject in need thereof, based upon the expression level of at least one biomarker or an expression pattern of at least one biomarker that is associated with NSTEMI or STEMI.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or 0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, at least about 70%, at least about 80%, or at least about 90% of corresponding positions in each of the molecules are occupied by nucleotides which normally base pair with each other (e.g., A:T and G:C nucleotide pairs).

As used herein, an “immunoassay” refers to any binding assay that uses an antibody capable of binding specifically to a target molecule to detect and quantify the target molecule.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, the term “diagnosis” refers to the determination of the presence of a disease or disorder, such as NSTEMI or STEMI. In some embodiments of the present invention, methods for making a diagnosis are provided which permit determination of the presence of a disease or disorder, such as NSTEMI or STEMI.

The terms “dysregulated” and “dysregulation” as used herein describes a decreased (down-regulated) or increased (up-regulated) level of expression of a mRNA, protein, or other molecule present and detected in a sample obtained from a subject as compared to the level of expression of that mRNA, protein, or other molecule present in a comparator sample, such as a comparator sample obtained from one or more normal subjects, not-at-risk subjects, or from the same subject at a different time point. In some instances, the level of mRNA, protein, or other molecule expression is compared with an average value obtained from more than one not-at-risk individuals. In other instances, the level of mRNA, protein, or other molecule expression is compared with a mRNA, protein, or other molecule level assessed in a sample obtained from one normal, not-at-risk subject.

As used herein, the terms “therapy” or “therapeutic regimen” refer to those activities taken to alleviate or alter a disorder or disease, such as a myocardial infarction, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

An “effective amount” as used herein, means an amount which provides a therapeutic or prophylactic benefit.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease, such as a myocardial infarction, being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder, such as a myocardial infarction, experienced by a subject.

As used herein, “Differentially expressed level” refers to a group of biomarkers having differentially decreased expression level, or a differentially elevated expression level. It also means that in a group of biomarkers some biomarkers have differentially decreased expression and some biomarkers have differentially elevated expression.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a comparator.

As used herein “endogenous” refers to any material from or produced inside an organism, cell, tissue or system.

The term “expression” as used herein is defined as the transcription and/or translation of a particular nucleotide sequence.

“Fragment” as the term is used herein, is a nucleic acid sequence that differs in length (i.e., in the number of nucleotides) from the length of a reference nucleic acid sequence, but retains essential properties of the reference molecule. For example, the fragment may be at least about 50% of the length of the reference nucleic acid sequence. More exemplary, the fragment may be at least about 75% of the length of the reference nucleic acid sequence. Even more exemplary, the fragment is at least about 95% of the length of the reference nucleic acid sequence.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic animals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

As used herein, “hybridization,” “hybridize(s)” or “capable of hybridizing” is understood to mean the forming of a double or triple stranded molecule or a molecule with partial double or triple stranded nature. Complementary sequences in the nucleic acids pair with each other to form a double helix. The resulting double-stranded nucleic acid is a “hybrid.” Hybridization may be between, for example two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids (e.g., LNA compounds). One or both of the nucleic acids may be immobilized on a solid support. Hybridization techniques may be used to detect and isolate specific sequences, measure homology, or define other characteristics of one or both strands. The stability of a hybrid depends on a variety of factors including the length of complementarity, the presence of mismatches within the complementary region, the temperature and the concentration of salt in the reaction or nucleotide modifications in one of the two strands of the hybrid. Hybridizations are usually performed under stringent conditions, for example, at a salt concentration of no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for probe hybridizations. In a particularly exemplary embodiment, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

The term “inhibit,” as used herein, means to suppress or block an activity or function by at least about ten percent relative to a control value. For example, the activity may be suppressed or blocked by 50% compared to a control value, more exemplary by 75%, and even more exemplary by 95%.

As used herein, an “instructional material” includes a publication, a recording, a diagram, or any other medium of expression which can be used to communicate the usefulness of a compound, composition, vector, method or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient or recipients.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, “microRNA” or “miRNA” describes small non-coding RNA molecules, generally about 15 to about 50 nucleotides in length, for example, 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which may be a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Synthetic mutant” includes any purposefully generated mutant or variant protein or nucleic acid. Such mutants can be generated by, for example, chemical mutagenesis, polymerase chain reaction (PCR) based approaches, or primer-based mutagenesis strategies well known to those skilled in the art.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by the invention include, but are not restricted to, oligonucleotides, nucleic acids, antibodies, cell membrane receptors, monoclonal antibodies and antisera reactive with specific antigenic determinants (such as on viruses, cells or other materials), drugs, peptides, cofactors, lectins, sugars, polysaccharides, cells, cellular membranes, and organelles. Targets are sometimes referred to in the art as anti-probes.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to the discovery that the level of expression or expression pattern of particular biomarkers is associated with particular forms of myocardial infarction. In some embodiments, one or more biomarkers associated with myocardial infarction is up-regulated, or expressed at a higher than normal level. In other embodiments, one or more biomarkers associated with myocardial infarction is down-regulated, or expressed at a lower than normal level. Thus, the invention relates to compositions and methods useful for the diagnosis, assessment, and characterization of myocardial infarction in a subject in need thereof, based upon the expression level or expression pattern of one or more biomarkers that is associated with myocardial infarction.

In various embodiments, the methods of the invention relate to methods of treating a myocardial infarction in a subject, where the subject has been identified as having a differentially expressed level of one or more biomarkers associated with a myocardial infarction, is administered a therapy. In some embodiments, the methods of the invention relate to altering a treatment of myocardial infarction in a subject who has been identified as not having a differentially expressed level of one or more biomarkers associated with a myocardial infarction, by discontinuing administration of a therapy.

In some embodiments, the myocardial infarction is ST-segment elevation myocardial infarction (STEMI). In some embodiments, the myocardial infarction is non-ST-segment elevation myocardial infarction (NSTEMI). In various embodiments of the compositions and methods of the invention described herein, biomarkers associated with myocardial infarction is at least one of MMP9, TIMP1, P-selectin, phosphorylated ERK5 (P-ERK5), ERK5, TxB2, and any combination thereof.

### Assays and Methods of Diagnosis

In some embodiments, the invention relates to a screening assay of a subject to determine whether the subject has a differentially expressed level or pattern of one or more biomarkers disclosed herein. In some embodiments, the invention relates to a screening assay of a subject to determine whether the subject has an elevated level of expression of one or more biomarkers selected from the group MMP9, TIMP1, P-selectin, phosphorylated ERK5 (P-ERK5), ERK5, TxB2, and any combination thereof. In other embodiments, the invention relates to a screening assay of a subject to determine whether the subject has a reduced level of expression of one or more biomarkers selected from the group MMP9, TIMP1, P-selectin, phosphorylated ERK5 (P-ERK5), ERK5, TxB2, and any combination thereof. In one embodiment, the biomarker is a gene. The present invention provides methods of assessing the level of gene products, including mRNAs and polypeptides, in a subject. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof.

In one embodiment, the invention is a diagnostic assay for diagnosing a myocardial infarction in a subject, by determining whether the subject has a differentially expressed level of one or more biomarkers disclosed herein. In one embodiment, the invention is a diagnostic assay for diagnosing a myocardial infarction in a subject, by determining whether the subject has an elevated level of expression of one or more biomarkers selected from the group MMP9, TIMP1, P-selectin, phosphorylated ERK5 (P-ERK5), ERK5, TxB2, and any combination thereof. In another embodiment, the invention is a diagnostic assay for diagnosing a myocardial infarction in a subject, by determining whether the subject has a reduced level of expression of one or more biomarkers selected from the group MMP9, TIMP1, P-selectin, phosphorylated ERK5 (P-ERK5), ERK5, TxB2, and any combination thereof. In yet another embodiment, the invention is a diagnostic assay for diagnosing a myocardial infarction in a subject, by determining whether the subject has both a reduced level of expression of one or more biomarkers and an elevated level of expression of one or more biomarkers. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof.

In various embodiments, to determine whether the level of expression of one or more biomarkers disclosed herein is increased or reduced in a biological sample of the subject, the level of expression of the one or more biomarkers disclosed herein is compared with the level of the one or more biomarkers of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In some embodiments, the invention is a method of determining whether the subject has a differentially expressed level of one or more biomarkers disclosed herein. In some embodiments, the invention is a method of determining whether the subject has an elevated level of expression one or more biomarkers disclosed herein. In other embodiments, the invention is a method of determining whether a subject has a reduced level of expression of one or more biomarkers disclosed herein. The present invention provides methods of assessing the level of gene products, including mRNAs and polypeptides, in a subject. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of one or more biomarkers selected from the group MMP9, TIMP1, P-selectin, phosphorylated ERK5 (P-ERK5), ERK5, TxB2, and any combination thereof. In some embodiments, the biological sample is blood, plasma, saliva, or urine, or a component thereof.

In one embodiment, the invention is a method of diagnosing a myocardial infarction in a subject, by determining whether the subject has a differentially expressed level of one or more biomarkers disclosed herein. In one embodiment, the invention is a method of diagnosing a myocardial infarction in a subject, by determining whether the subject has an elevated level of expression of one or more biomarkers disclosed herein. In another embodiment, the invention is a method of diagnosing a myocardial infarction in a subject, by determining whether the subject has a reduced level of expression of one or more biomarkers disclosed herein. In various embodiments, the level of a gene product in the biological sample can be determined by assessing the amount of polypeptide present in the biological sample, the amount of mRNA present in the biological sample, the amount of activity of the gene product in the biological sample, the amount of binding activity of the gene product in the biological sample, or a combination thereof. In various embodiments, the gene product is a gene product of one or more biomarkers selected from the group MMP9, TIMP1, P-selectin, phosphorylated ERK5 (P-ERK5), ERK5, TxB2, and any combination thereof.

In various embodiments, to determine whether the level of expression of one or more biomarkers disclosed herein is increased or reduced in a biological sample of the subject, the level of expression of the at least one gene disclosed herein is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject.

In various embodiments of the assays of the invention, the level of expression of the biomarker is determined to be elevated or increased when the level of expression of the biomarker is increased by less than 10%, at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, at least 600%, at least 700%, at least 800%, at least 900%, at least 1000%, at least 1500%, at least 2000%, at least 2500%, at least 3000%, at least 4000%, at least 5000%, or more, when compared with a comparator control.

In various embodiments of the methods of the invention, the level of expression of the biomarker is determined to be elevated or increased when the level of expression of the biomarker in the biological sample is increased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In various embodiments of the methods of the invention, the level of expression of the biomarker is determined to be elevated or increased when the level of expression of the biomarker in the biological sample is increased by less than 1 units, at least 1 units, at least 2 units, at least 3 units, at least 4 units, at least 5 units, at least 6 units, at least 7 units, at least 8 units, at least 9 units, at least 10 units, at least 20 units, at least 30 units, at least 40 units, at least 50 units, at least 60 units, at least 70 units, at least 80 units, at least 90 units, at least 100 units, at least 150 units, at least 200 units, at least 250 units, at least 500 units, at least 1000 units, or more units, when compared with a comparator, wherein units may be an expression of concentration (i.e., pg/mL, ng/mL, μg/mL, mg/mL, g/mL, etc.) or an expression of mass (i.e., pg, ng, μg, mg, g, etc.).

In other various embodiments of the assays of the invention, the level of expression of the biomarker is determined to be reduced or decreased when the level of expression of the biomarker is reduced or decreased by less than 10%, at least 10%, at least 20%, at least 30%, at least 40%, at least 50%, at least 60%, at least 70%, at least 80%, at least 90%, at least 100%, at least 125%, at least 150%, at least 175%, at least 200%, at least 250%, at least 300%, at least 400%, at least 500%, at least 600%, at least 700%, at least 800%, at least 900%, at least 1000%, at least 1500%, at least 2000%, at least 2500%, at least 3000%, at least 4000%, at least 5000%, or more, when compared with a comparator control.

In other various embodiments of the assays of the invention, the level of expression of the biomarker is determined to be reduced or decreased when the level of expression of the biomarker is reduced or decreased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In various embodiments of the methods of the invention, the level of expression of the biomarker is determined to be reduced or decreased when the level of expression of the biomarker in the biological sample is decreased by less than 1 units, at least 1 units, at least 2 units, at least 3 units, at least 4 units, at least 5 units, at least 6 units, at least 7 units, at least 8 units, at least 9 units, at least 10 units, at least 20 units, at least 30 units, at least 40 units, at least 50 units, at least 60 units, at least 70 units, at least 80 units, at least 90 units, at least 100 units, at least 150 units, at least 200 units, at least 250 units, at least 500 units, at least 1000 units, or more units, when compared with a comparator, wherein units may be an expression of concentration (i.e., pg/mL, ng/mL, μg/mL, mg/mL, g/mL, etc.) or an expression of mass (i.e., pg, ng, μg, mg, g, etc.).

In various embodiments of the methods of the invention, the subject is diagnosed with STEMI when the level of MMP9 is elevated when compared with a comparator. In one embodiment, the comparator is the level of MMP9 in a subject or population with NSTEMI. In one embodiment, the comparator is the level of MMP9 in a healthy subject or population.

In one embodiment, the subject is diagnosed with STEMI when the level of MMP9 is elevated by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the subject is diagnosed with STEMI when the level of MMP9 is elevated by about 2.4 fold relative to a healthy control subject or population.

In one embodiment, the subject is diagnosed with STEMI when the level of MMP9 is elevated by about 1.5 fold relative to a subject or population with NSTEMI.

In one embodiment, the level of MMP9 is elevated from about 284 ng/mL in a control sample to about 638 ng/mL in a sample of a subject with STEMI. In one embodiment, the level of MMP9 is elevated from about 395 ng/mL in a sample of a subject with NSTEMI to about 638 ng/mL in a sample of a subject with STEMI.

In various embodiments of the methods of the invention, the subject is diagnosed with NSTEMI when the level of MMP9 is decreased when compared with a comparator. In one embodiment, the comparator is the level of MMP9 in a subject or population with STEMI.

In one embodiment, the subject is diagnosed with NSTEMI when the level of MMP9 is decreased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the subject is diagnosed with NSTEMI when the level of MMP9 is decreased by about 1.5 fold relative to a subject or population with STEMI.

In one embodiment, the level of MMP9 is decreased from about 638 ng/mL in a sample of a subject with STEMI to about 395 ng/mL in a sample of a subject with NSTEMI.

In various embodiments of the methods of the invention, the subject is diagnosed with NSTEMI when the level of MMP9 is elevated when compared with a comparator. In one embodiment, the comparator is the level of MMP9 in a healthy subject or population.

In one embodiment, the subject is diagnosed with NSTEMI when the level of MMP9 is elevated by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the subject is diagnosed with NSTEMI when the level of MMP9 is elevated by about 1.4 fold relative to a healthy control subject or population.

In one embodiment, the level of MMP9 is elevated from about 284 ng/mL in a control sample to about 395 ng/mL in a sample of a subject with NSTEMI.

In various embodiments of the methods of the invention, the subject is diagnosed with STEMI when the level of TIMP1 is decreased when compared with a comparator. In one embodiment, the comparator is the level of TIMP1 in a subject or population with NSTEMI. In one embodiment, the comparator is the level of TIMP1 in a healthy subject or population.

In one embodiment, the subject is diagnosed with STEMI when the level of TIMP1 is decreased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator. In one embodiment, the subject is diagnosed with STEMI when the level of TIMP1 is decreased by about 1.1 fold when compared with a comparator. In one embodiment, the level of TIMP1 is decreased from about 326 ng/mL in a sample of a subject with NSTEMI to about 288 ng/mL in a sample of a subject with STEMI. In one embodiment, the level of TIMP1 is decreased from about 404 ng/mL in a control subject to about 288 ng/mL in a sample of a subject with STEMI.

In various embodiments of the methods of the invention, the subject is diagnosed with NSTEMI when the level of TIMP1 is elevated when compared with a comparator. In one embodiment, the comparator comprises the level of TIMP1 in a subject or population with STEMI.

In one embodiment, the subject is diagnosed with NSTEMI when the level of TIMP1 is elevated by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator. In one embodiment, the subject is diagnosed with NSTEMI when the level of TIMP1 is elevated by about 1.1 fold when compared with a comparator. In one embodiment the level of TIMP1 is elevated from about 288 ng/mL in a sample of a subject with STEMI to about 326 ng/mL in a sample of a subject with NSTEMI.

In various embodiments of the methods of the invention, the subject is diagnosed with NSTEMI when the level of TIMP1 is decreased when compared with a comparator. In one embodiment, the comparator comprises the level of TIMP1 in a healthy subject or population.

In one embodiment, the subject is diagnosed with NSTEMI when the level of TIMP1 is decreased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator. In one embodiment, the subject is diagnosed with NSTEMI when the level of TIMP1 is decreased by about 1.2 fold when compared with a comparator. In one embodiment the level of TIMP1 is decreased from about 404 ng/mL in a control sample to about 326 ng/mL in a sample of a subject with NSTEMI.

In one embodiment, the subject is diagnosed with STEMI when the level of MMP9 is elevated as compared to a comparator, and the level of TIMP1 is decreased as compared to a comparator. In one embodiment, the subject is diagnosed with STEMI when the level of MMP9 is elevated as compared to the level in a subject or population with NSTEMI, and the level of TIMP1 is decreased as compared to the level in a subject or population with NSTEMI. In one embodiment, the subject is diagnosed with STEMI when the ratio of MMP9/TIMP1 is increased as compared to the ratio of MMP9/TIMP1 in a subject or population with NSTEMI.

In one embodiment, the subject is diagnosed with NSTEMI when the level of MMP9 is decreased as compared to a comparator, and the level of TIMP1 is elevated as compared to a comparator. In one embodiment, the subject is diagnosed with NSTEMI when the level of MMP9 is decreased as compared to the level in a subject or population with STEMI, and the level of TIMP1 is elevated as compared to the level in a subject or population with STEMI. In one embodiment, the subject is diagnosed with STEMI when the ratio of MMP9/TIMP1 is decreased as compared to the ratio of MMP9/TIMP1 in a subject or population with STEMI.

In one embodiment, the method comprises using a multi-dimensional nonlinear algorithm to determine if the expression level of a set of biomarkers in the biological sample is statistically different than the expression level in a control sample. In various embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In the assay methods of the invention, a test biological sample from a subject is assessed for the level of expression of one or more biomarkers disclosed herein in the biological sample obtained from the patient. The level of expression of the biomarker in the biological sample can be determined by assessing the amount of polypeptide gene product of the gene in the biological sample, the amount of mRNA gene product of the gene in the biological sample, the amount of activity of the gene product in the biological sample, the amount of binding activity of the gene product in the biological sample, or a combination thereof.

In various embodiments, the subject is a human subject, and may be of any race, sex and age. Representative subjects include those who are suspected of having a myocardial infarction, those who have been diagnosed with myocardial infarction or those who are at risk of developing myocardial infarction. In some embodiments, the subject is suspected of having, or has been diagnosed as having, ST-segment elevation myocardial infarction (STEMI) or non-ST-segment elevation myocardial infarction (NSTEMI).

In various embodiments, the test sample is a biological sample (e.g., fluid, tissue, cell, cellular component, etc.) of the subject containing at least a fragment of a gene product (e.g., polypeptide or mRNA) of one or more biomarkers disclosed herein. The biological sample can be a sample from any source which contains a polypeptide or a nucleic acid, such as a fluid, tissue, cell, cellular component, or a combination thereof. A biological sample can be obtained by appropriate methods, such as, by way of examples, blood draw, fluid draw, or biopsy. A biological sample can be used as the test sample; alternatively, a biological sample can be processed to enhance access to the polypeptides or nucleic acids, or copies of the nucleic acids, and the processed biological sample can then be used as the test sample. For example, in various embodiments, nucleic acid (e.g., mRNA, cDNA prepared from mRNA, etc.) is prepared from a biological sample, for use in the assays and methods. Alternatively, or in addition, if desired, an amplification method can be used to amplify nucleic acids comprising all or a fragment of an mRNA in a biological sample, for use as the test sample in the assessment of the level in the biological sample. In some embodiments, the biological sample is blood, plasma, saliva, or urine, or a component thereof. In another embodiment, the biological sample is blood, or a component thereof.

In various embodiments of the invention, methods of measuring polypeptide levels in a biological sample obtained from a patient include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods useful for the detection, identification and measurement of nucleic acids in the methods of the invention include high throughput RNA and DNA sequencing.

In some embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In one embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequences in the biological sample obtained from a subject can be used to determine the level a nucleic acid in the biological sample obtained from a subject. The array of oligonucleotide probes can be used to determine the level of target nucleic acid alone, or the level of the target nucleic acid in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of 32P, 33P, 35S or 3H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a biological sample, including a biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. In one embodiment, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In one embodiment, in the Southern blot, the nucleic acid probe is labeled with a tag. That tag can be a radioactive isotope, a fluorescent dye or the other well-known materials. Another type of process for the specific detection of nucleic acids in a biological sample known in the art are the hybridization methods as exemplified by U.S. Pat. Nos. 6,159,693 and 6,270,974, and related patents. To briefly summarize one of those methods, a nucleic acid probe of at least 10 nucleotides, for example at least 15 nucleotides, or at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. In one embodiment, the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In one embodiment, the process for determining the quantitative and qualitative profile of the nucleic acid of interest according to the present invention is characterized in that the amplifications are real-time amplifications performed using a labeled probe, for example a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. For example, the Tm for any one of the hydrolysis-probes of the present invention is in the range of about 67° C. to about 70° C. Most exemplary, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. For example, a primer featured in the invention includes a nucleotide sequence sufficiently complementary to hybridize to a nucleic acid sequence of about 12 to 25 nucleotides. More exemplary, the primer differs by no more than 1, 2, or 3 nucleotides from the target flanking nucleotide sequence. In another aspect, the length of the primer can vary in length. In one embodiment, the length of the primer is about 15 to 28 nucleotides in length (e.g., 16, 17, 18, 19, 20, 21, 22, 23, 24, 25, 26, or 27 nucleotides in length).

### Treatments

In one aspect, the invention provides a method of treating a myocardial infarction in a subject. For example, in certain embodiments, the subject has been identified as having a differentially expressed level of one or more biomarkers disclosed herein. In one embodiment, the method comprises administering to the subject an effective amount of a therapy or therapeutic composition disclosed herein or known in the art of myocardial infarction treatment.

Additionally, therapeutic agents suitable for administration to a particular subject can be identified by detecting one or more biomarkers disclosed herein. Accordingly, treatments or therapeutic regimens for use in subjects having, suspected of having, or at risk of having a myocardial infarction can be selected based on the level of one or more biomarkers disclosed herein, and compared to a reference value. In various embodiments, a recommendation is made regarding whether to initiate, or continue administration of a therapy. In some embodiments, a recommendation is made regarding whether to discontinue administration of a therapy.

Any drug or combination of drugs disclosed herein or known in the art may be administered to a subject to treat a disease. The drug or drugs can be formulated in any number of ways, often according to various formulations known in the art or as disclosed or referenced herein.

In various embodiments, any drug or combination of drugs disclosed herein, or known in the art, is not administered to a subject to treat a myocardial infarction. In these embodiments, the practitioner may refrain from administering the drug or combination of drugs, may recommend that the subject not be administered the drug or combination of drugs, or may prevent the subject from being administered the drug or combination of drugs.

In various embodiments, one or more additional drugs may be optionally administered in addition to those that are recommended or have been administered. An additional drug will typically not be any drug that is not recommended or that should be avoided.

In various embodiments, the treatment administered is at least one selected from the group including administration of one or more blood thinners, administration of one or more thrombolytic drugs, administration of one or more PAR1 antagonists, angioplasty, stent placement, coronary artery bypass surgery, angioplasty, stent placement, administration of one or more thrombolytic drugs, administration of one or more thromboxane receptor antagonists, administration of one or more P2Y₁₂ receptor antagonists, and any combination thereof.

In one embodiment, the subject is diagnosed with STEMI, and is treated with at least one treatment selected from the group including administration of one or more blood thinners, administration of one or more thrombolytic drugs, administration of one or more PAR1 antagonists, angioplasty, stent placement, coronary artery bypass surgery, angioplasty, stent placement, administration of one or more thrombolytic drugs, administration of one or more thromboxane receptor antagonists, administration of one or more P2Y₁₂ receptor antagonists, and any combination thereof.

In one embodiment, the treatment or therapy is administered to a subject diagnosed with STEMI, where the subject is diagnosed with STEMI when the level of MMP9 is elevated by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the treatment or therapy is administered to a subject diagnosed with STEMI, where the subject is diagnosed with STEMI when the level of MMP9 is elevated by about 2.4 fold relative to a healthy control subject or population.

In one embodiment, the treatment or therapy is administered to a subject diagnosed with STEMI, where the subject is diagnosed with STEMI when the level of MMP9 is elevated by about 1.5 fold relative to a subject or population with NSTEMI.

In one embodiment, the subject is diagnosed with NSTEMI, and is treated with at least one treatment selected from the group including administration of one or more blood thinners, administration of one or more thrombolytic drugs, administration of one or more PAR1 antagonists, angioplasty, stent placement, coronary artery bypass surgery, angioplasty, stent placement, administration of one or more thrombolytic drugs, administration of one or more thromboxane receptor antagonists, administration of one or more P2Y₁₂ receptor antagonists, and any combination thereof.

In one embodiment, the treatment or therapy is administered to a subject diagnosed with NSTEMI, where the subject is diagnosed with NSTEMI when the level of MMP9 is decreased when compared with a comparator. In one embodiment, the comparator is the level of MMP9 in a subject or population with STEMI.

In one embodiment, the treatment or therapy is administered to a subject diagnosed with NSTEMI, where the subject is diagnosed with NSTEMI when the level of MMP9 is decreased by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the treatment or therapy is administered to a subject diagnosed with NSTEMI, where the subject is diagnosed with NSTEMI when the level of MMP9 is decreased by about 1.5 fold relative to a subject or population with STEMI.

In one embodiment, the treatment or therapy is administered to a subject diagnosed with NSTEMI, where subject is diagnosed with NSTEMI when the level of MMP9 is elevated when compared with a comparator. In one embodiment, the comparator is the level of MMP9 in a healthy subject or population.

In one embodiment, the treatment or therapy is administered to a subject diagnosed with NSTEMI, where the subject is diagnosed with NSTEMI when the level of MMP9 is elevated by less than 1 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, at least 1000 fold, or more, when compared with a comparator.

In one embodiment, the treatment or therapy is administered to a subject diagnosed with NSTEMI, where the subject is diagnosed with NSTEMI when the level of MMP9 is elevated by about 1.4 fold relative to a healthy control subject or population.

In one embodiment, the subject is diagnosed with STEMI, and is not treated with at least one treatment selected from the group including administration of one or more blood thinners, administration of one or more thrombolytic drugs, administration of one or more PAR1 antagonists, angioplasty, stent placement, coronary artery bypass surgery, angioplasty, stent placement, administration of one or more thrombolytic drugs, administration of one or more thromboxane receptor antagonists, administration of one or more P2Y₁₂ receptor antagonists, and any combination thereof.

In one embodiment, the subject is diagnosed with NSTEMI, and is not treated with at least one treatment selected from the group including administration of one or more blood thinners, administration of one or more thrombolytic drugs, administration of one or more PAR1 antagonists, angioplasty, stent placement, coronary artery bypass surgery, angioplasty, stent placement, administration of one or more thrombolytic drugs, administration of one or more thromboxane receptor antagonists, administration of one or more P2Y₁₂ receptor antagonists, and any combination thereof.

### Kits

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample. In another embodiment, the kit comprises components useful for the quantification of a desired polypeptide in a biological sample. In a further embodiment, the kit comprises components useful for the assessment of the activity (e.g., enzymatic activity, substrate binding activity, etc.) of a desired polypeptide in a biological sample.

In one embodiment, the kit comprises a reagent for measuring the level of one or more biomarkers in a biological sample of a subject. For example, in one embodiment, the one or more biomarkers comprises at least one of MMP9, TIMP1, P-selectin, phosphorylated ERK5 (P-ERK5), ERK5, TxB2, and any combination thereof. In some embodiments, the biological sample is blood, urine, saliva or plasma, or a component thereof.

In a further embodiment, the kit comprises the components of an assay for selecting a treatment to be administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Platelet Dysregulation Following Myocardial Infarction

Platelets from patients with ST-segment elevation myocardial infarction (STEMI) and non-ST-segment elevation myocardial infarction (NSTEMI) were compared to platelets from healthy volunteers. Isolated platelets from patients with STEMI and NSTEMI were examined for surface receptor activation by flow cytometry. Post-receptor signal transduction pathways were assessed by Western blotting and zymography. Platelet-derived plasma biomarkers were evaluated by Receiver Operator Characteristic (ROC) analyses. Maximum platelet activation through the thromboxane receptor was greater in STEMI compared to NSTEMI (6-fold vs. 3.4-fold, respectively, p=0.007) but less through PAR1 (4.6-fold vs. 7.1-fold, respectively, p=0.001). Platelet Extracellular-signal related-kinase 5 (ERK5) activation was increased (2.2-fold over control platelets for STEMI, p=0.03 and 4.1-fold over control platelets for NSTEMI, p=0.009). Matrix metalloproteinase 9 (MMP9) content and enzymatic activity were several fold greater in platelets with M.I. compared to control. Mean plasma MMP9 concentration in patients with M.I. distinguishes between STEMI and NSTEMI (AUC 75% [C.I. 60-91], p=0.006), and is superior to troponin T (AUC 66% [C.I. 48-85], p=0.080), predicting STEMI with sensitivity 80% (95% C.I. 56-94), specificity 90% (C.I. 68-99), AUC 70% (C.I. 54-86, p<0.0001), and NSTEMI with sensitivity 50% (C.I. 27-70), specificity 90% (C.I. 68-99), AUC 70% (C.I. 54-86, p=0.03). Platelets from patients with STEMI and NSTEMI show differences in platelet receptor activation and post-receptor signaling, suggest the healthy platelet phenotype in which anti-platelet agents are designed is fundamentally different.

To examine mechanisms in platelets that may explain differences in outcomes in patients with STEMI compared to NSTEMI, blood from patients presenting with STEMI and NSTEMI in the emergency department (ED) following aspirin monotherapy was collected, and platelet receptor signaling and post-receptor signal transduction pathways were examined, and these data were compared to data obtained from platelets isolated from healthy individuals. Only platelet receptor agonists for which oral anti-platelet agents exert an effect (U46619 for the thromboxane receptor, ADP for the P2Y₁₂ receptor, and TRAP6 for PAR1) were utilized. It was examined whether platelet phenotype and hence platelet activation are different in patients with STEMI compared to those with NSTEMI. These studies can aid the understanding of dysregulated platelet activity observed in some patients following M.I.

The materials and methods employed in these experiments are now described.

### Study Population and Design

This study included 61784 (patients with M.I.) and 53791 (healthy subjects). The recruitment, consent, and blood collection procedures for STEMI, NSTEMI, and control patients are as follows: a STEMI alert is triggered by acute ST-segment elevation in two contiguous leads of the 12-lead ECG in the ambulance or immediately upon arrival in the ED. Delayed consent was granted for patients presenting with STEMI to avoid interfering with the door-to-balloon time. Each subject had four plasma citrate tubes of venous blood drawn within 10 minutes of arriving in the emergency department and before P2Y₁₂ antagonists were administered. This first sample was used for platelet isolation. The patients were then consented within 24 hours of blood draw after recovery from moderate sedation following coronary angiography and their sample was discarded if the patient declined to enroll and sign the consent form. For patients with NSTEMI, subjects were already in the ED or hospital for 1-10 hours and identified by elevated plasma cardiac troponin (plasma value greater than the 99th percentile of a healthy population with a 10% assay CV) with concomitant symptoms consistent with cardiac chest pain. Patients with NSTEMI were approached for consent prior to P2Y₁₂ antagonists being given or coronary angiography being conducted. Each subject had four plasma citrate tubes of blood drawn, and this was used for platelet isolation. Each patient was treated with 325 mg aspirin in the ambulance, in the ED, or hospital least 30 minutes prior to blood draw. Any patient who could not provide consent even if a surrogate was available was excluded, and patients with thrombocytopenia, anemia, or with active hematologic malignancies were excluded. Patients who were administered a loading dose of a P2Y₁₂ receptor antagonist in the pre-hospital setting or the ED prior to the blood draw were excluded. Healthy volunteer subjects were enrolled as a control group and were free from vascular disease and not taking vasoactive or anti-platelet medications. 60 subjects were enrolled: 20 healthy subjects and 40 patients with acute M.I. (20 with STEMI, 20 with NSTEMI). Basic patient demographics were recorded in a confidential manner and stored in a secure fashion with the inventor. Platelet function was studied within 1 hour of blood draw. To enhance the clinical relevance of this study, the summed agonist dose-response curves were generated only for platelet signaling pathways for which oral antagonists are available (aspirin for cyclooxygenase inhibition which decreases platelet thromboxane release and subsequent platelet thromboxane receptor stimulation, U46619 is the agonist used in this study; clopidogrel, ticagrelor, and prasugrel for P2Y₁₂ receptor inhibition, 2 methyl-ADP [ADP] is the agonist used in this study; vorapaxar for PAR1 inhibition, Thrombin Receptor Activator for Peptide 6 [TRAP] is the agonist used in this study).

### Platelet Function

For each study subject, blood was obtained by a trained medical professional into citrate plasma tubes, then centrifuged in a tabletop centrifuge at 1100 rpm for 15 mins. The platelet rich plasma (PRP) well above the buffy coat was decanted and the final platelet centrifugation step at 2600 rpm for 5 mins was conducted with a final concentration of 10 μM PGI2. The final washed platelet pellet from one human plasma citrate tube was resuspended in 1000 μL of fresh Tyrode's solution, which was diluted 1:20 in fresh Tyrode's solution. This was aliquoted into 100 μL quadruplicate platelet samples and stimulated with 1 μL of each drug concentration. After 15 minutes, 1 μL of labeled CD62P (p-selectin) antibody was incubated in the dark for 30 minutes. This reaction was then stopped by adding 100 μL of 2% formalin to each reaction, and then quantification of platelet surface P-selectin was made possible using an Accuri Flow Cytometer (BD Biosciences) at 10K events per sample. Data was then processed through FloJo (Ashland, Oreg.). Platelet surface P-selectin was used as marker of platelet activation (alpha granule exocytosis and increased platelet surface expression in response to pharmacologic doses of platelet receptor agonists: 2-methyl ADP, PAR1, and U46619). Platelet surface p-selectin was quantified in quadruplicate using the geometric mean. For each concentration of drug for each patient, the sample was run in quadruplicate (baseline, four increasing doses of agonist=20 samples per agonist) for each of three agonists per patient, for one patient encounter.

### Reagents

TRAP6 and Thrombin (Cayman Chemicals), 2-methyl-ADP (Tocris, Bristol, UK), U46619 (Cayman Chemical), gelatin (Fisher Scientific). ERK5 antibody #12950, p-ERK5 antibody #3371, MMP9 antibody #13667, CD41 antibody #13807, CD45 antibody #13917, actin antibody #4970 (all from Cell Signaling Technology), TIMP1 antibody #ab61224 (Abcam), GAPDH antibody #sc-25778 (Santa Cruz Biotech.). CD62P-PE antibody Clone AK4 #12-0628-62 (eBioscience/ThermoFisher Waltham, Mass.). CD41-FITC antibody #303703 (BioLegend, San Diego, Calif.). CD45-PE antibody #12-9459-42 (eBioscience/ThermoFisher Waltham, Mass.). Anti-mouse and anti-rabbit secondary antibody (GE healthcare, UK).

### Biochemistry

Platelet Protein Studies

Cell lysis and cell protein extraction, SDS PAGE, and Western blotting were conducted using buffers and techniques as described previously (Cameron S J, et al., 2015, Circulation, 132:47-58). Blocking buffer was 3% BSA (Sigma Aldrich) dissolved in Tris-buffered saline at pH 8.0 (Fisher Scientific) with 0.1% Tween-20 (TBS-T) at room temperature for 1 hour. Primary antibody was 1:1000 overnight at 4° C. in 3% BSA/TBS-T. Secondary antibody (GE Healthcare, Buckinghamshire, UK) was used in a 1:2000 titer in 5% milk/TBS-T for 1 hour at room temperature. ECL reagent used was Supersignal West Pico (Thermo Scientific) for each antibody. Final autoradiographic films (Bioblot BXR, Laboratory Product Sales, Rochester N.Y.) were quantified by densitometry using ImageJ software (NIH).

MMP Activity Assay

Platelets were isolated as described above and placed in 50% vol/vol 2× non-denaturing sample buffer at the following final concentration: Tris-HCl 250 mM, 0.5% SDS, 1% glycerol, 0.05% bromophenol blue for 10 minutes without boiling and separated by SDS-PAGE (12% bis-acrylamide containing 1 mg/mL final concentration gelatin within the matrix) at 125 V (constant voltage, room temperature). The gel was renatured by gently rocking in 2.5% Triton-X-100 for 30 minutes at room temperature, then decanting, and incubating in fresh zymogram buffer for 12 hours overnight at 37° C. The zymogram buffer was decanted, and the gel was rocked at room temperature for 4 hours in Simply Blue Safestain (Invitrogen). MMP activity was noted by clear bands in the final gel (a reverse image). Total MMP activity in each lane was quantified by densitometry using ImageJ software (NIH).

Immuno-Depletion of White Blood Cells from PRP

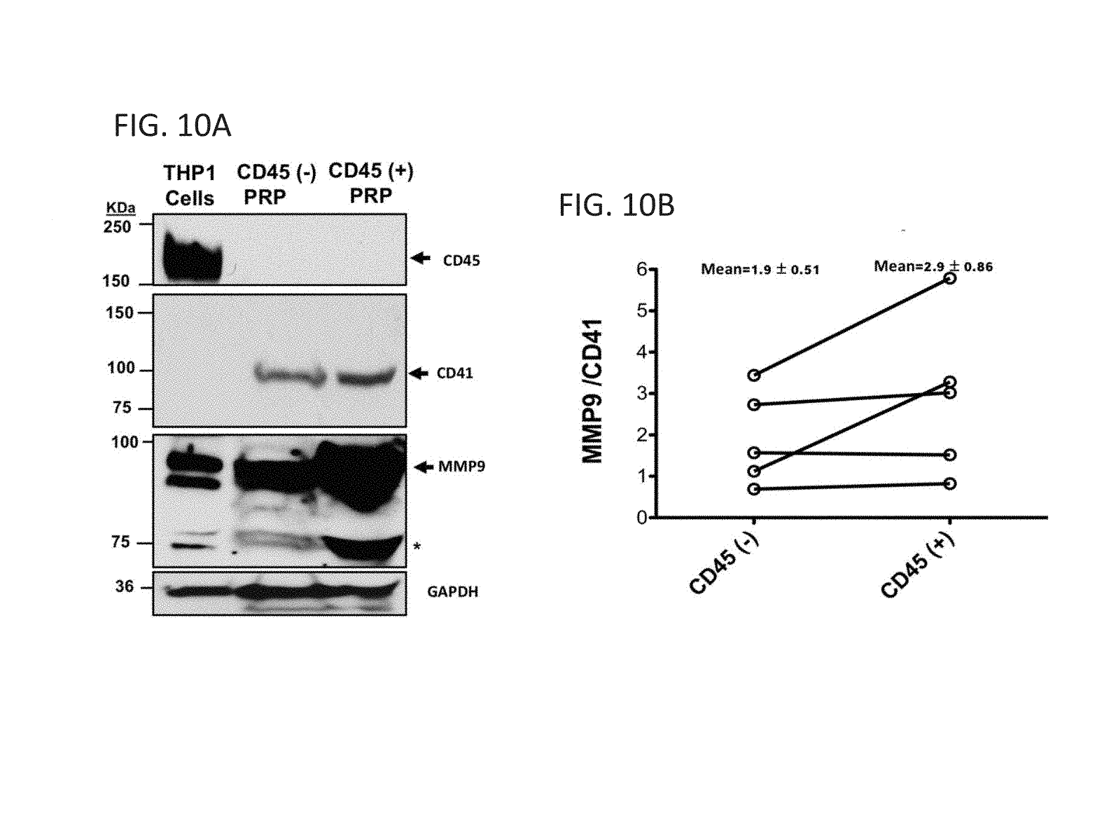

PRP was obtained as above, with quality control studies taken to stay above the buffy coat to decrease the number of white blood cell (WBC) contaminants. A FITC-tagged CD41 antibody was used as a platelet-specific marker and a PE-tagged CD45 antibody as a WBC-specific marker, using flow cytometry for isolated platelet analysis. The final step of each PRP isolate for agonist stimulation was 100 μL in volume with a mean platelet count of 9954±5 and a mean WBC count of 12±1 (average WBC contamination of PRP=0.12%). For quality control, the final PRP isolate was divided into two equal volumes with one half incubated with a human anti-CD45 antibody to deplete residual WBCs using the MagniSort™ Human CD45 Depletion Kit (Invitrogen #8804-6802-74) according to the manufacturer's instructions. A THP1 cell line which is known to contain MMP9 and CD45 was used as a positive control for WBCs (Vandooren J, et al., 2017, PLoS One, 12:e0174853; Favier B, et al., 2003, J Immunol, 171:5027-33). CD45 but not CD41 immunoreactivity was observed in THP1. Platelet MMP9 protein content was normalized to immunoreactive CD41 in CD45-depleted, CD45(+), and control IgG non-depleted, CD45(−), PRP. A slight but not significant decrease in platelet MMP9 immunoreactivity was observed (MMP9/CD41 ratio 1.9±0.51 in depleted vs. 2.9±0.86 in non-depleted PRP, P=0.36, FIG. 10A-FIG. 10B).

### Immunoassay

Enzyme-linked immunosorbant assays (ELISAs) were conducted according to the manufacturer's instructions using the sandwich ELISA technique. The ELISAs for plasma MMP9 (cat #DY911) and TIMP1 (cat# DY970-05) were purchased from R&D Systems (Minneapolis, Minn.). The ELISA for plasma thromboxane B2 (cat#501020) was purchased from Cayman Chemicals.

### Statistical Analysis

Data were analyzed by using GraphPad Prism 7 (GraphPad Software, Inc., La Jolla, Calif.). Dichotomous clinical variables are presented as frequencies. Continuous clinical variables are presented as mean with standard error of the mean (SEM) unless otherwise stated. Differences between groups were analyzed with ANOVA for repeated measurements with Bonfer-roni post-test or pairwise comparisons between the baseline and experimental condition was made using the t-test. Plasma biomarker data was evaluated by ROC curve analysis with performance of the biomarker for predicting the clinical condition reported as Area Under Curve (AUC), sensitivity, and specificity with 95% confidence interval.

The results of the experiments are now described.

### MMP9 is a Useful Biomarker for Predicting Myocardial Infarction

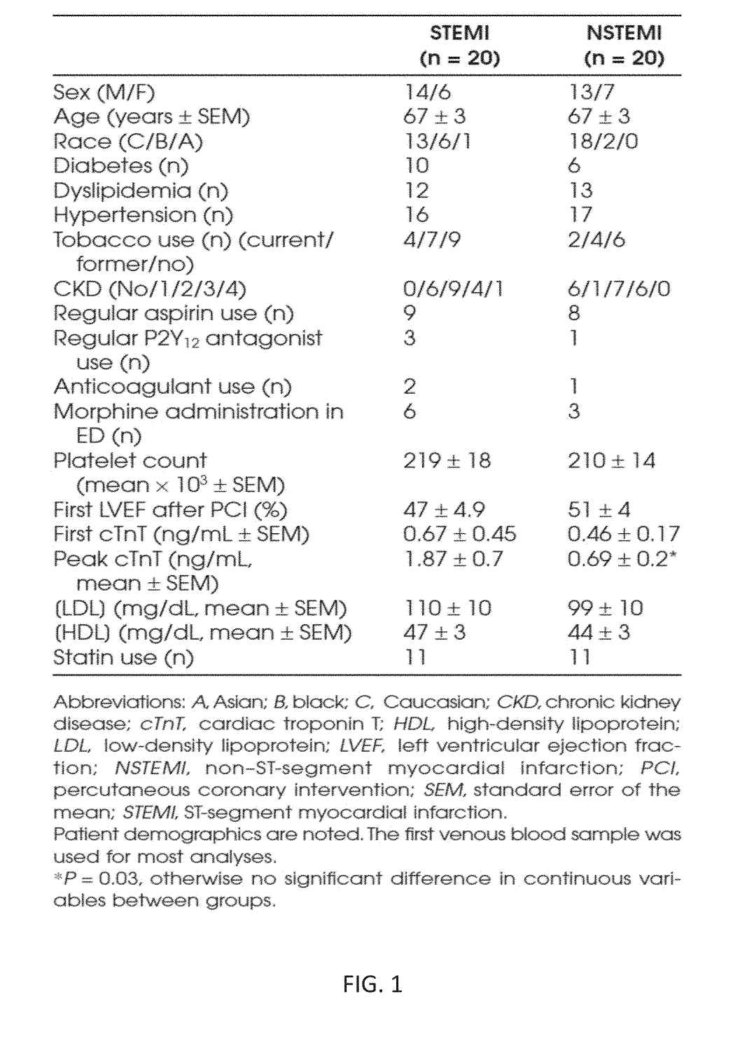

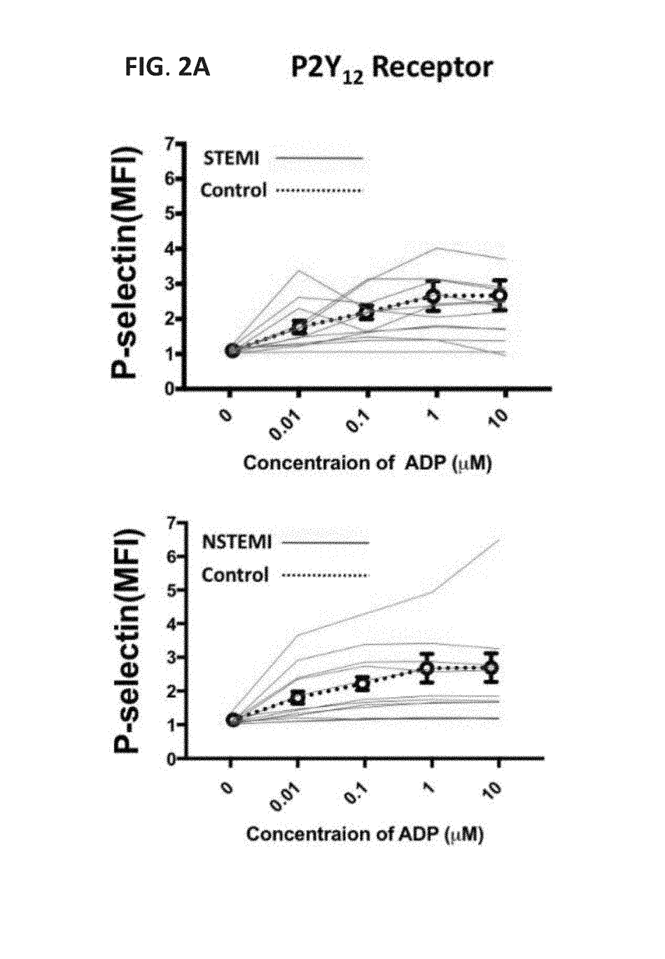

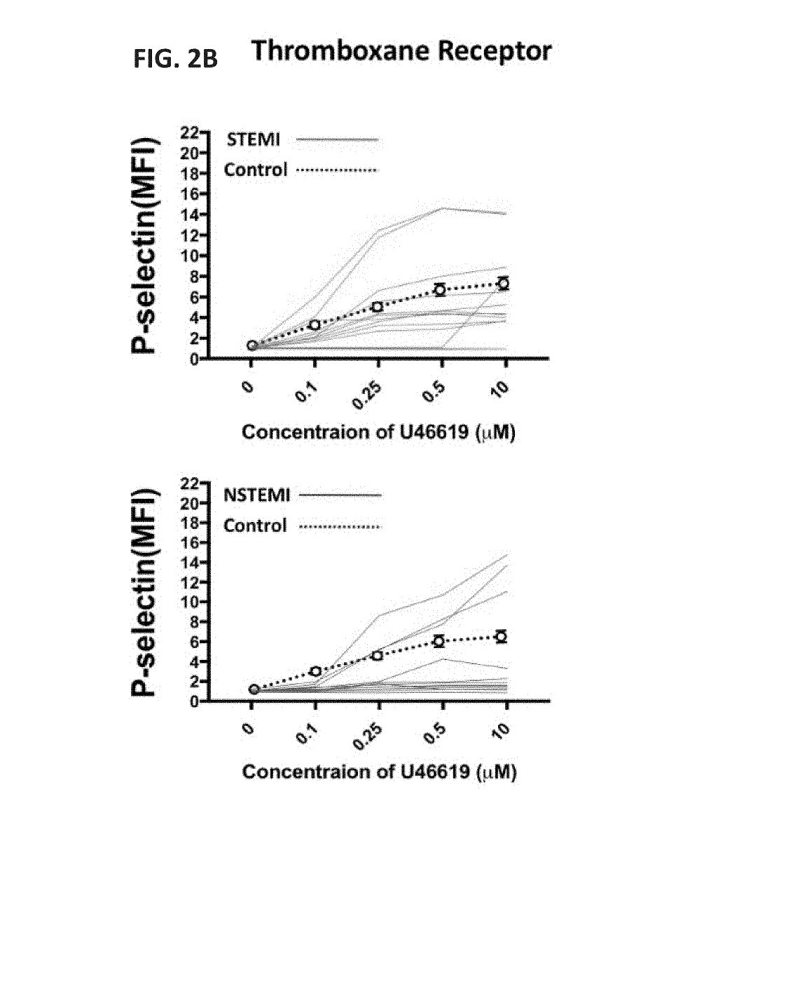

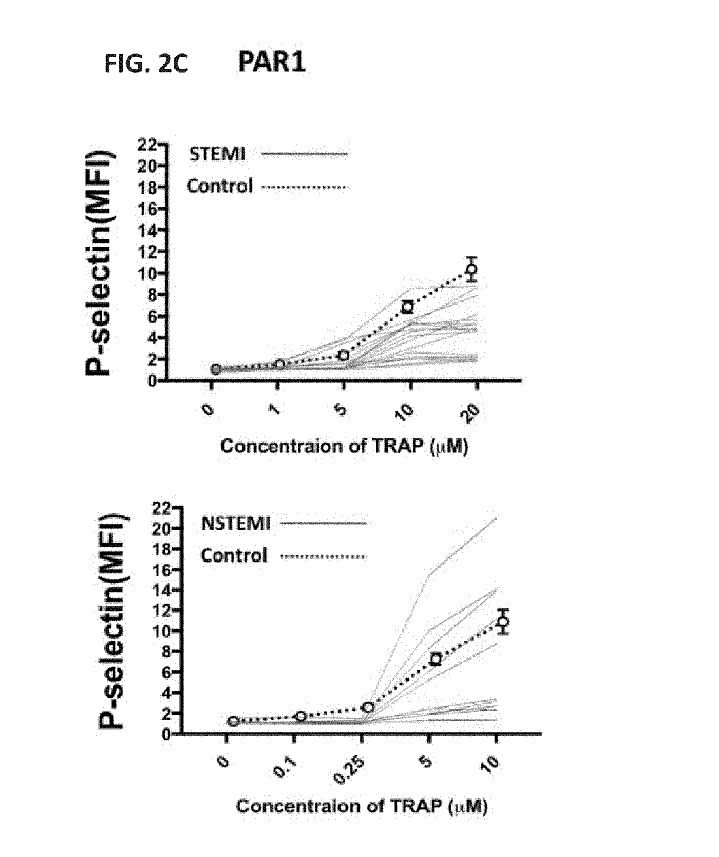

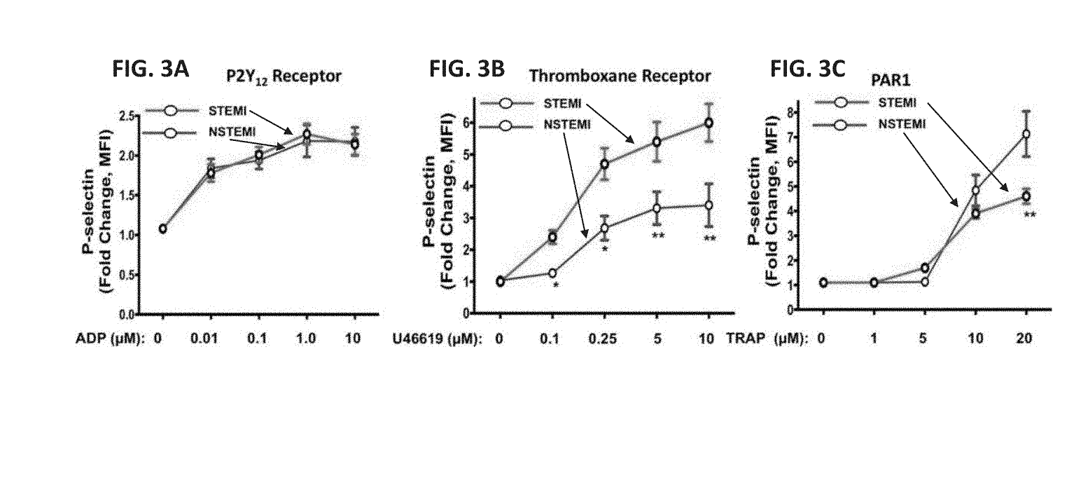

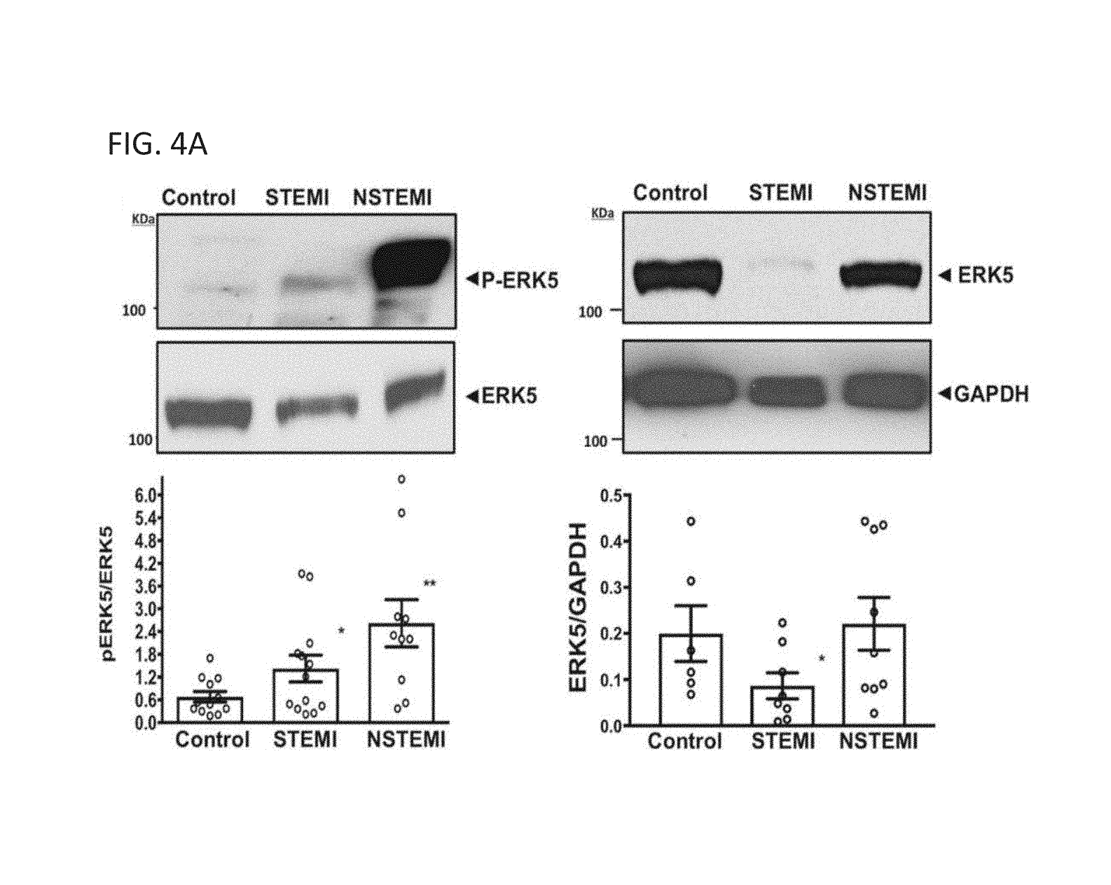

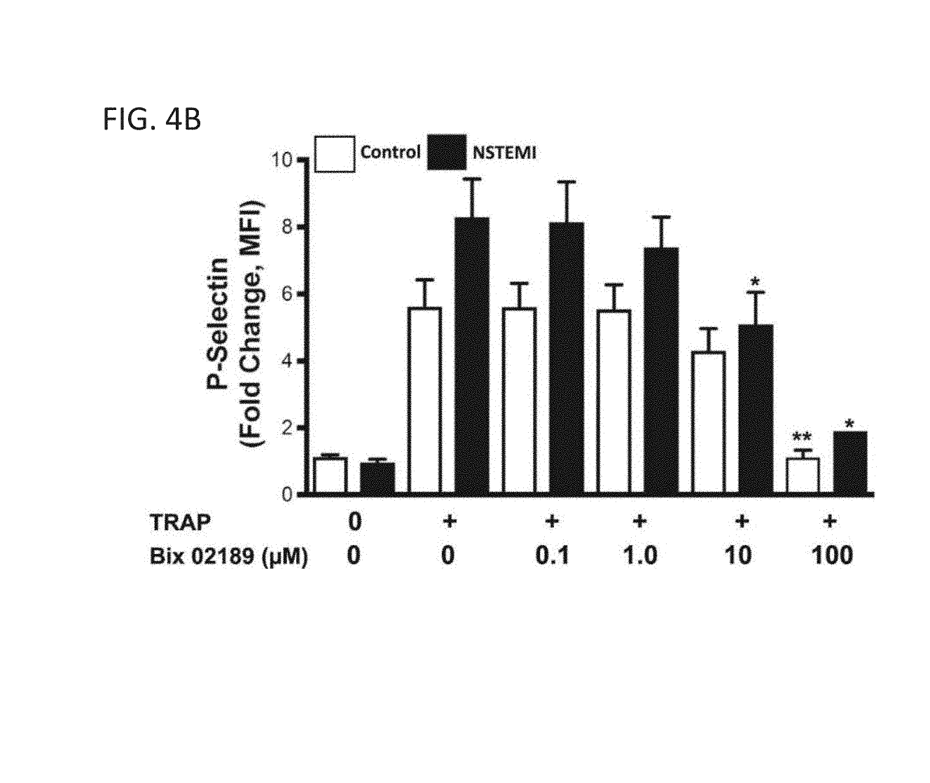

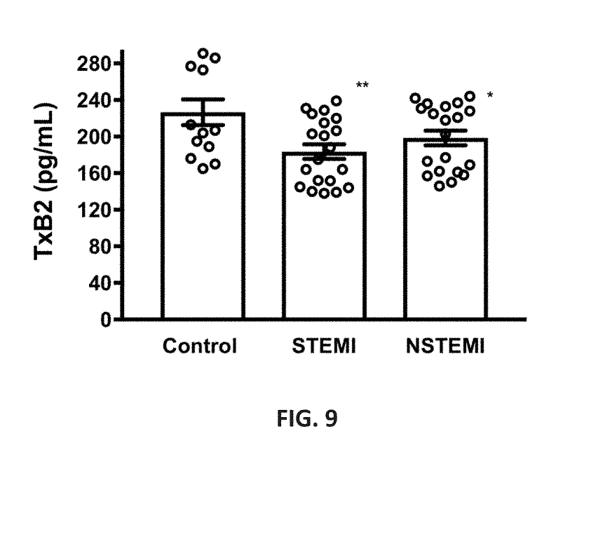

The characteristics of the patients in this example are indicated in FIG. 1. Demographic data, clinical variables, and cardiovascular co-morbidities were similar between patients with STEMI and NSTEMI with the notable exception that patients with STEMI had a greater peak cardiac troponin concentration. Using a well-validated method of flow cytometry to detect platelet surface p-selectin expression as an index of platelet activation (Schmitz G, et al., 1998, Thromb Haemost, 79:885-96), a wide range of individual platelet responses to surface receptor agonists was observed for patients presenting with STEMI and NSTEMI, in spite of each patient being treated with 325 mg aspirin at least 30 minutes before blood draw. For comparison, healthy volunteer agonist dose-response curves for platelet surface receptors are shown on the same graphs (FIG. 2A-FIG. 2C). No difference in P2Y₁₂ receptor signaling at any agonist concentration between platelets from patients with STEMI and NSTEMI was found, which revealed almost superimposable dose response curves for each group of patients (FIG. 3A). The platelet thromboxane receptor signaling pathway was more activated in patients presenting with STEMI compared to NSTEMI (FIG. 3B) while the platelet PAR1 signaling pathway was more activated in patients presenting with NSTEMI compared to STEMI (FIG. 3C). Using a thromboxane metabolite (thromboxane B2, TxB2) as a plasma marker of aspirin inhibition of platelet cyclooxygenase (COX), a decrease in plasma TxB2 concentration in patients presenting with STEMI and NSTEMI who had been treated with aspirin compared to healthy volunteers not treated with aspirin was observed (FIG. 9). Consistent with prior reports that platelet ERK5, which is a redox-activated kinase, promotes dysregulated platelet activity and myocardial infarct expansion involving MMP9 in a murine model (Cameron S J, et al., 2015, Circulation, 132:47-58), it was observed that platelet ERK5 activation, platelet MMP9 content, and platelet MMP9 enzymatic activity were changed in human platelets following M.I. Using a phospho-specific antibody which detects the ERK5 activation motif, a mild increase (2.2-fold over baseline) in platelet ERK5 activation was observed in patients presenting with STEMI and a marked increase (4.4-fold over baseline) in NSTEMI (FIG. 4A). Further, a concomitant 2.2-fold decrease in platelet ERK5 protein expression in patients presenting at the time of STEMI compared with NSTEMI was observed, likely explaining the difference in activation between groups (FIG. 4A). Using an inhibitor of ERK5, agonist-induced platelet activation in healthy platelets could be inhibited in a dose-dependent fashion, which was slightly more efficacious in platelets taken from patients with NSTEMI (FIG. 4B).

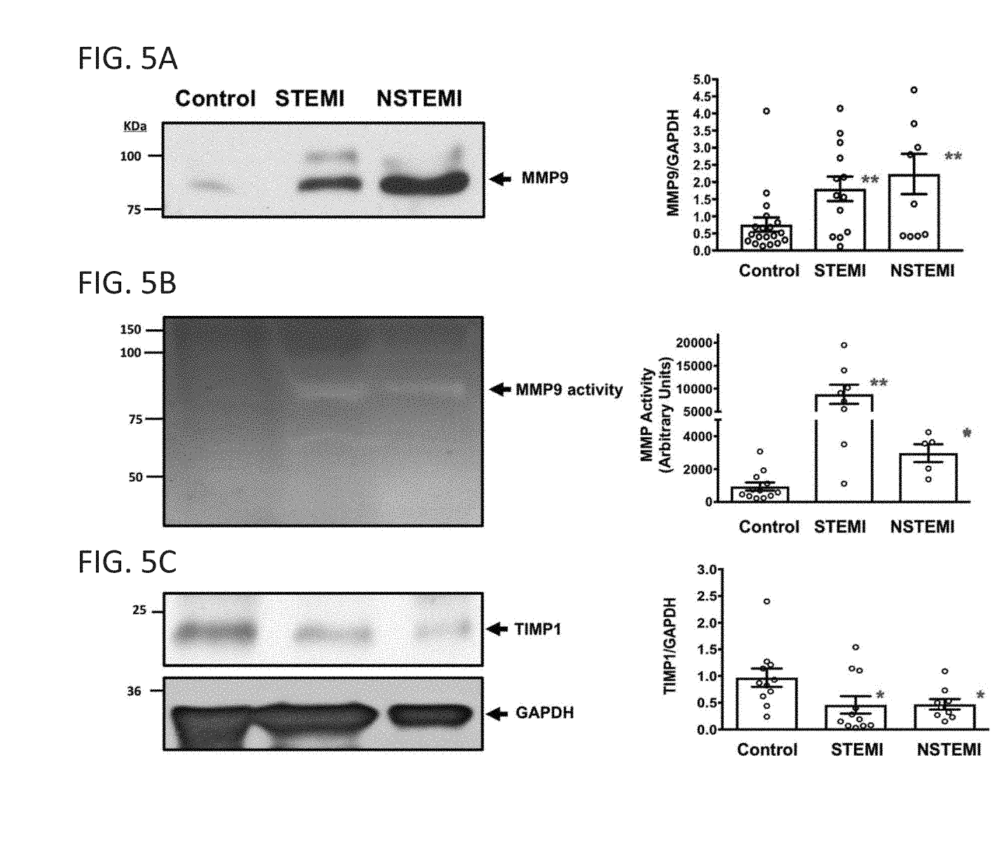

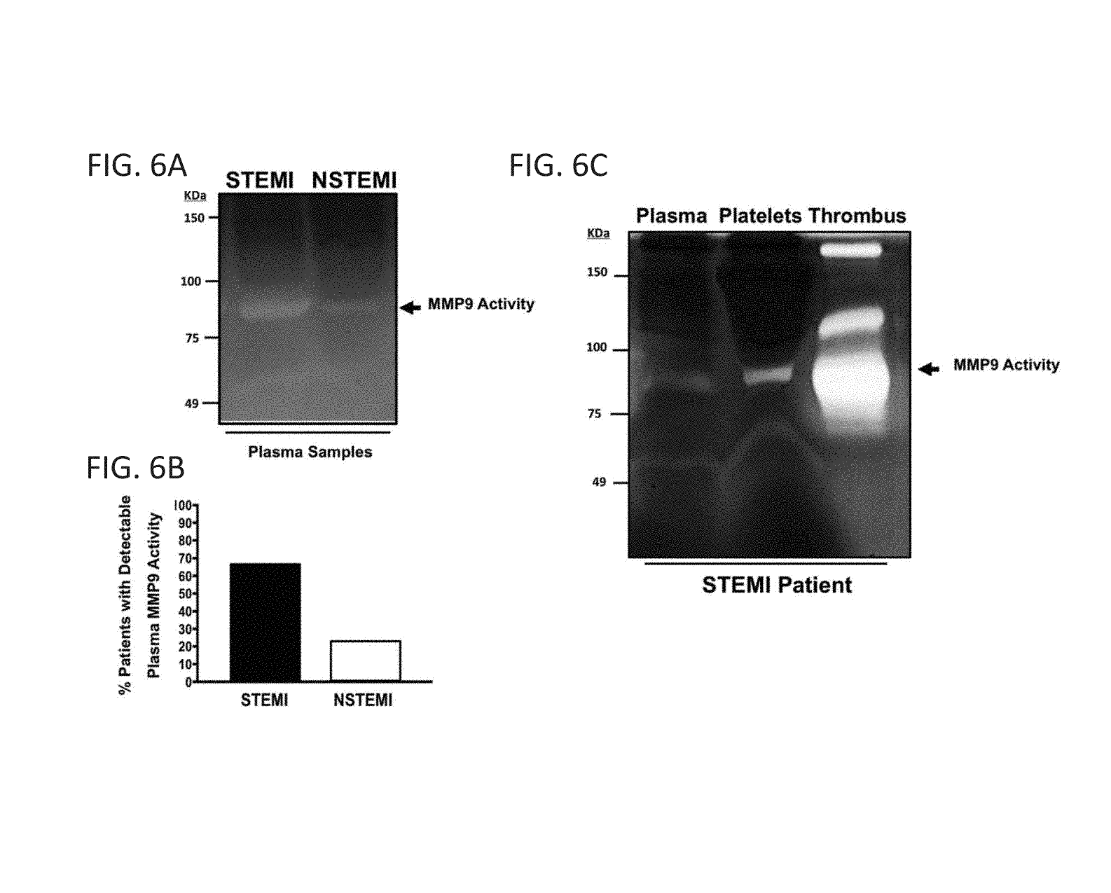

Platelets are enriched in matrix metalloproteinases (MMPs) (Seizer P, et al., 2013, Thromb Haemost, 110:903-9). MMPs may be important for platelet signaling and tissue remodeling at remote sites, including myocardial infarct extension and myocardial rupture (Cameron S J, et al., 2015, Circulation, 132:47-58; Matsumura S, et al., 2005, J Clin Invest, 115:599-609). MMP9 appears to be particularly important and is enriched in the coronary sinus of patients following M.I. (Higo S, et al., 2005, Circ J, 69:1180-5; Nishiguchi T, et al., 2016, Arterioscler Thromb Vasc Biol, 36:2460-2467) and has been implicated in deleterious cardiac remodeling in murine models of M.I. (Cameron S J, et al., 2015, Circulation, 132:47-58; He B J, et al., 2011, Nat Med, 17:1610-8), while platelet-mediated MMP2 release was reported to occur irrespective of aspirin treatment (Falcinelli E, et al., 2007, Br J Haematol, 138:221-30). Consistent with previous studies, it was observed that healthy human platelets express a negligible quantity of MMP9 protein (Mastenbroek T G, et al., 2015, Arterioscler Thromb Vasc Biol, 35:2554-61; Sheu J R, et al., 2004, Br J Pharmacol, 143:193-201). However, MMP9 protein content in platelet rich plasma persisted even after accounting for the possibility of occasional white blood cell contaminants (FIG. 10A-FIG. 10B). A marked upregulation in MMP9 protein expression in platelets from patients with STEMI and NSTEMI was observed (FIG. 5A). While MMP9 protein expression in platelets from STEMI and NSTEMI patients was similarly 3-fold higher than in healthy control platelets (FIG. 5A), MMP9 activity by zymography was 9-fold greater in STEMI and 3-fold greater in NSTEMI compared to control platelets suggesting different signaling properties in each may ultimately affect MMP9 activity (FIG. 5B). The reciprocal trend of less tissue inhibitor of MMP protein 1 (TIMP1) protein expression in platelets from patients with STEMI and NSTEMI compared to control was observed (0.46±0.16, 0.47±0.1, and 0.97±0.17, respectively) (FIG. 5C). Finally, MMP9 activity was detected in more plasma samples from patients with STEMI than NSTEMI which corresponded with the observation in platelets (FIG. 6A). Using plasma, isolated platelets, and aspirated intracoronary thrombus at the time of coronary intervention in a patient with an inferior STEMI, multiple enzymatically-active gelatinases were observed, though MMP9 activity appeared to be most prominent in coronary thrombus, aligning with the same band in platelets and plasma (FIG. 6C).

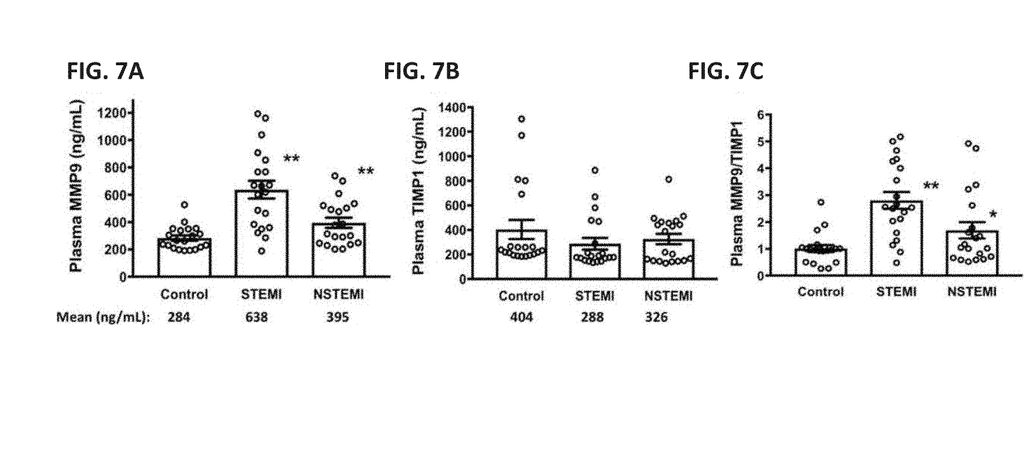

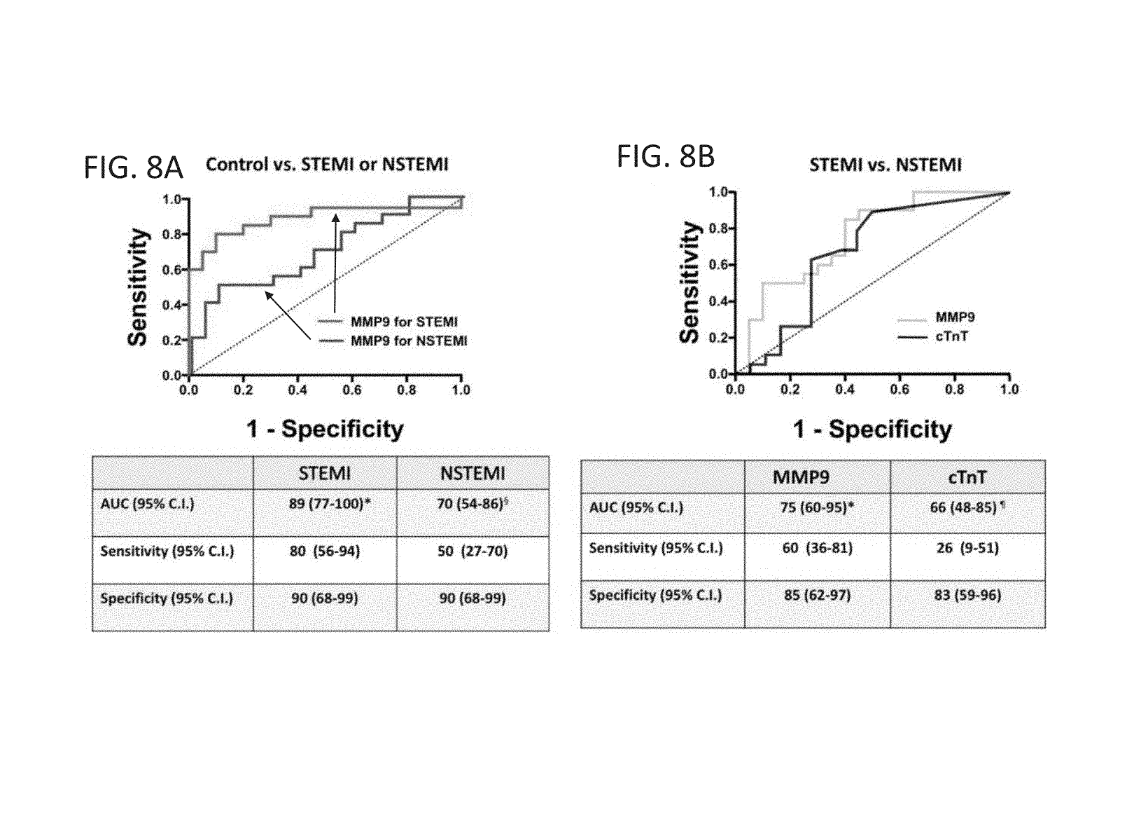

A stepwise increase in MMP9 concentration in the plasma of patients with NSTEMI and STEMI, respectively, compared to normal subjects. A reciprocal decrease in plasma TIMP1 expression in the same subjects was also observed (FIG. 7A-FIG. 7C). Using the first blood sample drawn from the patient on arrival, plasma MMP9 predicted STEMI with 90% specificity and 80% sensitivity and NSTEMI with 90% specificity and 50% sensitivity (FIG. 8A). Moreover, plasma MMP9 could distinguish between patients with STEMI and NSTEMI with 85% specificity and 60% sensitivity which was superior to cardiac troponin T from the same sample (FIG. 8B). Thus, MMP9, serving as an early platelet-derived-biomarker, is useful in predicting M.I. in an undifferentiated patient presenting with chest pain, with a non-acute ECG, and plasma cardiac biomarkers that are not yet released or less than the assay detection limit.

Platelet Phenotype in Patient with STEMI and NSTEMI

This is the first study to collect blood samples from STEMI and NSTEMI patients immediately upon arrival and immediately after administration of a loading dose of aspirin but no other anti-platelet agent. Platelets from patients presenting with STEMI were found to be significantly different from patients with NSTEMI with residual platelet activation observed following aspirin treatment in both groups, with different platelet receptor agonist sensitivity, and with different post-receptor signaling properties. Furthermore, platelets from patients with STEMI and NSTEMI were found to be phenotypically different from platelets in healthy volunteers in which most preclinical pharmacological studies for anti-platelet agents are conducted.

A significant advantage of the present study design was the provision of delayed patient consent for blood draw before loading with a P2Y₁₂ receptor antagonist and before coronary angiography. Both contribute significantly to experimental variability with respect to platelet function and biomarker evaluation which limits interpretation of the clinical data (Steinhubl S R., 2012, JACC Cardiovasc Interv, 5:278-80; Bhatt D L., 2004, J Am Coll Cardiol, 43:1127-9; Chen W H, et al., 2004, J Am Coll Cardiol, 43:1122-6). Furthermore, platelets were challenged with surface receptor agonists only for clinically-relevant signaling pathways for which oral antagonists are available (aspirin for the thromboxane pathway, vorapaxar for the PAR1 pathway, and clopidogrel, ticagrelor, and prasugrel for the P2Y₁₂ signaling pathway). This allowed insight into which platelet signaling pathways may be important in STEMI and NSTEMI. This opportunity may have been lost had aggregometry and examination of basal (unstimulated) platelet activation been relied on.

The biological processes involved in platelet activation and thrombosis in STEMI and NSTEMI are different, with STEMI vessel thrombosis usually manifesting as a large, flow-limiting occlusion of blood flow in an epicardial coronary artery. Atheroembolism, conversely, is thought to be the signature of platelet activation in NSTEMI and may be a recurrent phenomenon-sometimes apparent to the patient, sometimes undetected. Recurrent myocardial hypoperfusion events in NSTEMI was previously reported to be deleterious and linked to adverse long-term patient outcomes (Kuhl J T, et al., 2015, JACC Cardiovasc Imaging, 8:684-94). Hence, it stands to reason that uniformly treating patients with aspirin and a P2Y₁₂ receptor antagonist following STEMI and NSTEMI as though they were the same biological process might yield different long-term outcomes as reported (Chan M Y, et al., 2009, Circulation, 119:3110-7). The only anti-platelet agent administered at the time of blood draw for all the patients in this study was 325 mg aspirin. The platelet thromboxane signaling pathway which is inhibited by aspirin and activated by U46619 appears to be suppressed only in patients with NSTEMI in the present study. One may infer platelets from patients with STEMI are therefore more ‘aspirin resistant’. Since PAR1 signaling was significantly less active in platelets from the same aspirin-treated patients with STEMI compared to NSTEMI, these observations may reflect fundamental differences in platelet activation pathways in the peri-M.I. period.

The data indicate no differences in signaling through the P2Y₁₂ receptor for STEMI and NSTEMI platelets, but preference for PAR1 signaling in NSTEMI platelets, suggesting aspirin plus PAR1 blockade may be more beneficial for NSTEMI. It is therefore revealing that the Thrombin-Receptor Antagonist Vorapaxar in Acute Coronary Syndromes (TRACER) study, while showing a nonsignificant relative reduction of 8% in the primary end point was effectively triple therapy of DAPT plus vorapaxar, showed a trend toward clinically significant bleeding (Tricoci P, et al., 2012, N Engl J Med, 366:20-33). It is unclear whether a trial of aspirin plus a PAR1 antagonist or a PAR1 antagonist alone would change long-term patient outcomes in NSTEMI. A recent study by Wong et al. using a PAR4 antagonist elegantly demonstrated marked platelet inhibition without being blighted by the specter of bleeding diatheses common to PAR1 antagonists (Wong P C, et al., 2017, Sci Transl Med, 9(371)). This emphasizes the importance of returning to the fundamentals of platelet biology and understanding the nuances of platelet signaling to eventually see improvements in patient outcomes.

If platelet receptor function in platelets from STEMI and NSTEMI patients is different, it stands to reason that the post-receptor signal transduction molecules are also different. A downstream commonality between PAR1 and the thromboxane receptor which are both positively linked to the Gaq G-protein is the MAPK family member ERK5. Conversely, the P2Y₁₂ receptor appears not to distinguish between STEMI and STEMI platelet signaling in this example. This supports the prior observation in isolated human platelets and in a platelet-ERK5 deficient mouse that ERK5 activation is a requirement for human platelet PAR1 and thromboxane receptor, but not P2Y₁₂ receptor signaling (Cameron S J, et al., 2015, Circulation, 132:47-58). ERK5 has been demonstrated to activate platelets, particularly when in environments of redox stress such as may be expected in the peri-M.I. environment (Cameron S J, et al., 2015, Circulation, 132:47-58; Yang M, et al., 2017, Blood, 129:2917-2927; Cheng Z, et al., 2017, J Thromb Haemost, 15(8): 1679-1688). While the present example demonstrates an increase in platelet ERK5 activation in STEMI, the marked increase in activation in NSTEMI may be a downstream common mediator of and potential pharmacologic target of dysregulated platelet activity in thrombotic and ischemic heart disease. ERK5 tends to drive cell cycle progression in nucleated cells (Kato Y, et al., 1998, Nature, 395:713-6; Inesta-Vaquera F A, et al., 2010, Cell Signal, 22:1829-37), and so the presence in the anucleated platelet suggests a carryover remnant from the precursor nucleated megakaryocyte. By inference, ischemic cardiac disease may ‘reprogram’ bone marrow megakarycoytes to generate dysfunctional platelets with enhanced thrombotic potential, as has been suggested in both peripheral artery disease, diabetes, and atrial fibrillation (Hu L, et al., 2017, Circulation, 136(9): 817-833); Wisman P P, et al., 2015, PLoS One, 10:e0131356; Wysokinski W E, et al., 2017, Eur J Haematol, 98:615-621).

It is also demonstrated herein that platelets from patients with STEMI and NSTEMI, unlike platelets from healthy subjects, synthesize, and likely secrete activated MMP9 which has been reported to propagate coronary plaque rupture, tissue remodeling, and myocardial infarct expansion (Cameron S J, et al., 2015, Circulation, 132:47-58; He B J, et al., 2011, Nat Med, 17:1610-8; Zimmermann W H., 2015, J Am Coll Cardiol, 66:1375-7; Panizzi P, et al., 2010, J Am Coil Cardiol, 55:1629-38). Platelets are activated prior to coronary thrombosis and subsequent to myocardial necrosis, and are enriched in biomarkers. Platelets biomarkers therefore may be useful in early risk stratification (Ferroni P, et al., 2012, Thromb Haemost, 108:1109-23). This notion aligns with a previous study which showed coronary artery MMP9 concentration at the site of plaque rupture was an independent predictor of STEMI (Nishiguchi T, et al., 2016, Arterioscler Thromb Vasc Biol, 36:2460-2467).

This example shows that the signaling properties of platelets, platelet reactivity via specific surface receptors, and the platelet proteome are different in patients presenting acutely with STEMI and NSTEMI. The data also demonstrate that the platelet proteome could be exploited for biomarkers of prognostic value to predict M.I. since these proteins are often secreted prior to the patient sustaining a myocardial infarction event.

Platelets from patients with STEMI are not the same as those with NSTEMI, with differences in sensitivity to anti-platelet agents. Platelets contain potential biomarkers that can be utilized to predict coronary thrombosis before biomarkers for myocardial necrosis appear in the blood.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing ST-elevation myocardial infarction (STEMI) in a subject, the method comprising:
a. detecting the level of MMP9 in a biological sample obtained from the subject;
b. comparing the level of MMP9 in the biological sample to a control level of MMP9; and
c. diagnosing the subject with STEMI when MMP9 is elevated in the biological sample as compared to the control level.

2. The method of claim 1, wherein the biological sample comprises plasma.

3. The method of claim 1, wherein the subject is diagnosed with STEMI when the level of MMP9 in the sample is increased by 1.5-fold or greater as compared to the level of MMP9 in a subject or population having non-ST-elevated myocardial infarction (NSTEMI).

4. The method of claim 1, wherein the subject is diagnosed with STEMI when the level of MMP9 in the sample is increased by about 2.4 fold or greater as compared to the level of MMP9 in a healthy control subject or population not having myocardial infarction.

5. The method of claim 1, further comprising administering a STEMI treatment to the subject.

6. The method of claim 5, wherein the STEMI treatment comprises at least one treatment selected from the group consisting of angioplasty, stent placement, coronary artery bypass surgery, administration of one or more thrombolytic drugs, and administration of one or more thromboxane and P2Y₁₂ receptor antagonists.

7. A method of diagnosing non-ST-elevation myocardial infarction (NSTEMI) in a subject, the method comprising:
a. detecting the level of MMP9 in a biological sample obtained from the subject;
b. comparing the level of MMP9 in the biological sample to a control level of MMP9; and
c. diagnosing the subject with NSTEMI when MMP9 is differentially expressed in the biological sample as compared to the control level.

8. The method of claim 7, wherein the biological sample comprises plasma.

9. The method of claim 7, wherein the subject is diagnosed with NSTEMI when the level of MMP9 in the sample is decreased by greater than about 1.5-fold relative to the level of MMP9 in a subject or population having STEMI.

10. The method of claim 7, wherein the subject is diagnosed with NSTEMI when the level of MMP9 in the sample is increased by about 1.4 fold or greater as compared to the level of MMP9 in a healthy subject or population not having myocardial infarction.

11. The method of claim 7, wherein the subject is diagnosed with NSTEMI when a) the level of MMP9 in the sample is decreased by about 1.5 fold or greater as compared to the level of MMP9 in a subject or population having ST-elevated myocardial infarction (STEMI), and b) the level of MMP9 in the sample is increased by about 1.4 fold or greater as compared to the level of MMP9 in a healthy subject or population not having myocardial infarction.

12. The method of claim 7, further comprising administering a NSTEMI treatment to the subject.

13. The method of claim 12, wherein the NSTEMI treatment comprises at least one treatment selected from the group consisting of administration of one or more blood thinners, administration of one or more thromboxane, PAR1, or P2Y₁₂ receptor antagonists, angioplasty, stent placement, and coronary artery bypass surgery.

14. A kit for distinguishing between STEMI and NSTEMI, the kit comprising a reagent for measuring the level of MMP9 in a biological sample of a subject.

15. The kit of claim 14, wherein the biological sample comprises plasma.

16. A method of treating STEMI in a subject who has been identified as having a differentially expressed level MMP9, comprising administering an effective STEMI treatment to the subject.

17. The method of claim 16, wherein the differentially expressed level of MMP9 comprises at least one selected from the group consisting of:
a. an about 1.5 fold increase as compared to the level of MMP9 in a subject or population having non-ST-elevated myocardial infarction (NSTEMI); and
b. an about 2.4. fold increase as compared to the level of MMP9 in a healthy subject or population not having myocardial infarction.

18. A method of treating NSTEMI in a subject who has been identified as having a differentially expressed level of MMP9, comprising administering an effective NSTEMI treatment to the subject.

19. The method of claim 18, wherein the differentially expressed level of MMP9 comprises at least one selected from the group consisting of:
a. an about 1.5 fold decrease as compared to the level of MMP9 in a subject or population having ST-elevated myocardial infarction (STEMI); and
b. an about 1.4. fold increase as compared to the level of MMP9 in a healthy subject or population not having myocardial infarction.
